(19) United States
(12) Patent Application Publication
(10) Pub. No.: US 2022/0144966 A1
(43) Pub. Date: May 12, 2022

(54) Engineered Variant Antibodies that Bind CD38

(71) Applicant: Sorrento Therapeutics, Inc., San Diego, CA (US)

(72) Inventors: Xia Cao, San Diego, CA (US); Heyue Zhou, San Diego, CA (US); John Dixon Gray, San Diego, CA (US); Barbara A. Swanson, San Diego, CA (US)

(73) Assignee: Sorrento Therapeutics, Inc., San Diego, CA (US)

(21) Appl. No.: 17/599,377

(22) PCT Filed: Mar. 27, 2020
(86) PCT No.: PCT/US2020/025181

§ 371 Date: Sep. 28, 2021

Related U.S. Application Data
(60) Provisional application No. 62/825,983, filed on Mar. 29, 2019.

(51) Int. Cl. C07K 16/28

(52) U.S. Cl. CPC C07K 16/2896; C07K 2317/21; C07K 2317/94; C07K 2317/565; C07K 2317/732; C07K 2317/33

(57) ABSTRACT

The present disclosure provides anti-CD38 antigen-binding proteins such as fully human anti-CD38 IgG class antibodies each having an altered amino acid sequence in their heavy chain variable region and/or light chain variable region compared to their wild type parent antibody. The present disclosure provides engineered CD38 binding proteins, particularly anti-CD38 variant antibodies, or antigen-binding portions thereof, that specifically bind CD38, and uses thereof. The disclosed anti-CD38 antigen-binding proteins and antibodies have been engineered to exhibit improved characteristics compared to the parent antibody, such as improved binding to CD38 antigen, improved binding to CD38-expressing cells, and/or higher levels of cytotoxicity. The anti-CD38 variant antibodies can cross-react (bind) with cynomolgus CD38.

### CROSS-REFERENCE TO RELATED APPLICATIONS

This application claims the benefit of priority under 35 U.S.C. § 119 to U.S. provisional application No. 62/825,983, filed Mar. 29, 2019. The disclosures of the aforementioned application is incorporated by reference in its entirety.

Throughout this application various publications, patents, and/or patent applications are referenced. The disclosures of the publications, patents and/or patent applications are hereby incorporated by reference in their entireties into this application in order to more fully describe the state of the art to which this disclosure pertains. To the extent any material incorporated by reference conflicts with the express content of this application, the express content controls.

### TECHNICAL FIELD

The present disclosure provides anti-CD38 antigen-binding proteins such as fully human anti-CD38 IgG class antibodies each having an altered amino acid sequence in their heavy chain variable region and/or light chain variable region compared to their wild type parent antibody. Disclosed variant antibodies exhibit improved antigen binding, cell binding and cytotoxicity capabilities compared to their parent antibody. Disclosed variant antibodies also exhibit the same species cross reactivity as their parent antibody.

### BACKGROUND

CD38 is a 45 kD type II transmembrane glycoprotein with a long C-terminal extracellular domain and a short N-terminal cytoplasmic domain. The CD38 protein is a bifunctional ectoenzyme that can catalyze the conversion of NAD⁺ into cyclic ADP-ribose (cADPR) and also hydrolyze cADPR into ADP-ribose. During ontogeny, CD38 appears on CD34⁺ committed stem cells and lineage-committed progenitors of lymphoid, erythroid and myeloid cells. CD38 expression persists mostly in the lymphoid lineage with varying expression levels at different stages of T and B cell development.

CD38 is upregulated in many hematopoeitic malignancies and in cell lines derived from various hematopoietic malignancies, including non-Hodgkin's lymphoma (NHL), Burkitt's lymphoma (BL), multiple myeloma (MM), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), and chronic myeloid leukemia (CIVIL). On the other hand, most primitive pluripotent stem cells of the hematopoietic system are CD38⁻. CD38 expression in hematopoietic malignancies and its correlation with disease progression makes CD38 an attractive target for anti-CD38 antibody therapy.

CD38 has been reported to be involved in Ca²⁺ mobilization (Morra et al., 1998, FASEB J., 12: 581-592; Zilber et al., 2000, Proc. Natl. Acad. Sci. USA, 97: 2840-2845) and in the signal transduction through tyrosine phosphorylation of numerous signaling molecules, including phospholipase C-γ, ZAP-70, syk, and c-cbl, in lymphoid and myeloid cells or cell lines (Funaro et al., 1993, Eur. J. Immunol., 23: 2407-2411; Morra et al., 1998, FASEB J., 12: 581-592; Funaro et al., 1990, J Immunol, 145: 2390-2396; Zubiaur et al., 1997, J Immunol, 159: 193-205; Deaglio et al., 2003, Blood 102: 2146-2155; Todisco et al., 2000, Blood, 95: 535-542; Konopleva et al., 1998, 1 Immunol., 161: 4702-4708; Zilber et al., 2000, Proc. Natl. Acad. Sci. USA, 97: 2840-2845; Kitanaka et al., 1997, 1 Immunol., 159: 184-192; Kitanaka et al., 1999, 1 Immunol., 162: 1952-1958; Mallone et al., 2001, Int. Immunol., 13: 397-409). CD38 was proposed to be an important signaling molecule in the maturation and activation of lymphoid and myeloid cells during their normal development.

Evidence for the function of CD38 comes from CD38^{-/-} knockout mice, which have a defect in their innate immunity and a reduced T-cell dependent humoral response due to a defect in dendritic cell migration (Partida-Sanchez et al., 2004, Immunity, 20: 279-291; Partida-Sanchez et al., 2001, Nat. Med., 7:1209-1216). Nevertheless, it is not clear if the CD38 function in mice is identical to that in humans since the CD38 expression pattern during hematopoiesis differs greatly between human and mouse: a) unlike immature progenitor stem cells in humans, similar progenitor stem cells in mice express a high level of CD38 (Randall et al., 1996, Blood, 87:4057-4067; Dagher et al., 1998, Biol. Blood Marrow Transplant, 4:69-74), b) while during the human B cell development, high levels of CD38 expression are found in germinal center B cells and plasma cells (Uckun, 1990, Blood, 76:1908-1923; Kumagai et al., 1995, J Exp. Med., 181:1101-1110), in the mouse, the CD38 expression levels in the corresponding cells are low (Oliver et al., 1997, J. Immunol., 158:108-1115; Ridderstad and Tarlinton 1998, J. Immunol., 160:4688-4695).

Several anti-human CD38 antibodies with different proliferative properties on various tumor cells and cell lines have been described in the literature. For example, a chimeric OKT10 antibody with mouse Fab and human IgG1 Fc mediates antibody-dependent cell-mediated cytotoxicity (ADCC) very efficiently against lymphoma cells in the presence of peripheral blood mononuclear effector cells from either MM patients or normal individuals (Stevenson et al., 1991, Blood, 77:1071-1079). A CDR-grafted humanized version of the anti-CD38 antibody AT13/5 has been shown to have potent ADCC activity against CD38-positive cell lines. Human monoclonal anti-CD38 antibodies have been shown to mediate the in vitro killing of CD38-positive cell lines by ADCC and/or complement-dependent cytotoxicity (CDC), and to delay the tumor growth in SCID mice bearing MM cell line RPMI-8226 (WO2005/103083 A2). On the other hand, several anti-CD38 antibodies, IB4, SUN-4B7, and OKT10, but not D36, AT1, or AT2, induced the proliferation of peripheral blood mononuclear cells (PBMC) from normal individuals (Ausiello et al. 2000, Tissue Antigens, 56:539-547).

Some of the antibodies of the prior art have been reported to be able to trigger apoptosis in CD38⁺ B cells in a stroma cell-dependent or stroma-derived cytokine-dependent manner. An agonistic anti-CD38 antibody (IB4) has been reported to prevent apoptosis of human germinal center (GC) B cells (Zupo et al. 1994, Eur. J. Immunol., 24:1218-1222), and to induce proliferation of KG-1 and HL-60 AML cells (Konopleva et al. 1998, 1 Immunol., 161:4702-4708), but induces apoptosis in Jurkat T lymphoblastic cells (Morra et al. 1998, FASEB J., 12:581-592). Another anti-CD38 antibody, T16, induced apoptosis of immature lymphoid cells and leukemic lymphoblast cells from an ALL patient (Kumagai et al. 1995, 1 Exp. Med., 181:1101-1110), and of leukemic myeloblast cells from AML patients (Todisco et al. 2000, Blood, 95:535-542), but T16 induced apoptosis only in the presence of stroma cells or stroma-derived cytokines (IL-7, IL-3, stem cell factor).

There remains a need in the art for effective treatments based on CD38, particularly anti-CD38 antibodies. The present disclosure provides variant antibodies engineered to exhibit higher affinity binding to their target antigen and improved cell killing capabilities compared to the wild type parent antibody.

### SUMMARY

The present disclosure provides a n anti-CD38 antigen-binding protein or fully human anti-CD38 antibody, or an antigen-binding fragment thereof, comprising a heavy chain variable region and a light chain variable region, wherein the heavy chain variable region comprises a heavy chain complementarity determining region 1 (CDR1) a heavy chain CDR2 and a heavy chain CDR3, and the light chain variable region comprises a light chain CDR1, a light chain CDR2, and a light chain CDR3; and (a) the heavy chain CDR1 has the amino acid sequence of SEQ ID NO:29, the heavy chain CDR2 has the amino acid sequence of SEQ ID NO:30, the heavy chain CDR3 has the amino acid sequence of SEQ ID NO:31, the light chain CDR1 has the amino acid sequence of SEQ ID NO:32, the light chain CDR2 has the amino acid sequence of SEQ ID NO:33, and the light chain CDR3 has the amino acid sequence of SEQ ID NO:34; (b) the heavy chain CDR1 has the amino acid sequence of SEQ ID NO:35, the heavy chain CDR2 has the amino acid sequence of SEQ ID NO:36, the heavy chain CDR3 has the amino acid sequence of SEQ ID NO:37, the light chain CDR1 has the amino acid sequence of SEQ ID NO:38, the light chain CDR2 has the amino acid sequence of SEQ ID NO:39, and the light chain CDR3 has the amino acid sequence of SEQ ID NO:40; (c) the heavy chain CDR1 has the amino acid sequence of SEQ ID NO:41, the heavy chain CDR2 has the amino acid sequence of SEQ ID NO:42, the heavy chain CDR3 has the amino acid sequence of SEQ ID NO:43, the light chain CDR1 has the amino acid sequence of SEQ ID NO:44, the light chain CDR2 has the amino acid sequence of SEQ ID NO:45, and the light chain CDR3 has the amino acid sequence of SEQ ID NO:46; (d) the heavy chain CDR1 has the amino acid sequence of SEQ ID NO:47, the heavy chain CDR2 has the amino acid sequence of SEQ ID NO:48, the heavy chain CDR3 has the amino acid sequence of SEQ ID NO:49, the light chain CDR1 has the amino acid sequence of SEQ ID NO:50, the light chain CDR2 has the amino acid sequence of SEQ ID NO:51, and the light chain CDR3 has the amino acid sequence of SEQ ID NO:52; (e) the heavy chain CDR1 has the amino acid sequence of SEQ ID NO:53, the heavy chain CDR2 has the amino acid sequence of SEQ ID NO:54, the heavy chain CDR3 has the amino acid sequence of SEQ ID NO:55, the light chain CDR1 has the amino acid sequence of SEQ ID NO:56, the light chain CDR2 has the amino acid sequence of SEQ ID NO:57, and the light chain CDR3 has the amino acid sequence of SEQ ID NO:58; (f) the heavy chain CDR1 has the amino acid sequence of SEQ ID NO:59, the heavy chain CDR2 has the amino acid sequence of SEQ ID NO:60, the heavy chain CDR3 has the amino acid sequence of SEQ ID NO:61, the light chain CDR1 has the amino acid sequence of SEQ ID NO:62, the light chain CDR2 has the amino acid sequence of SEQ ID NO:63, and the light chain CDR3 has the amino acid sequence of SEQ ID NO:64; (g) the heavy chain CDR1 has the amino acid sequence of SEQ ID NO:65, the heavy chain CDR2 has the amino acid sequence of SEQ ID NO:66, the heavy chain CDR3 has the amino acid sequence of SEQ ID NO:67, the light chain CDR1 has the amino acid sequence of SEQ ID NO:68, the light chain CDR2 has the amino acid sequence of SEQ ID NO:69, and the light chain CDR3 has the amino acid sequence of SEQ ID NO:70; (h) the heavy chain CDR1 has the amino acid sequence of SEQ ID NO:71, the heavy chain CDR2 has the amino acid sequence of SEQ ID NO:72, the heavy chain CDR3 has the amino acid sequence of SEQ ID NO:73, the light chain CDR1 has the amino acid sequence of SEQ ID NO:74, the light chain CDR2 has the amino acid sequence of SEQ ID NO:75, and the light chain CDR3 has the amino acid sequence of SEQ ID NO:76; (i) the heavy chain CDR1 has the amino acid sequence of SEQ ID NO:77, the heavy chain CDR2 has the amino acid sequence of SEQ ID NO:78, the heavy chain CDR3 has the amino acid sequence of SEQ ID NO:79, the light chain CDR1 has the amino acid sequence of SEQ ID NO:80, the light chain CDR2 has the amino acid sequence of SEQ ID NO:81, and the light chain CDR3 has the amino acid sequence of SEQ ID NO:82; or (j) the heavy chain CDR1 has the amino acid sequence of SEQ ID NO:83, the heavy chain CDR2 has the amino acid sequence of SEQ ID NO:84, the heavy chain CDR3 has the amino acid sequence of SEQ ID NO:85, the light chain CDR1 has the amino acid sequence of SEQ ID NO:86, the light chain CDR2 has the amino acid sequence of SEQ ID NO:87, and the light chain CDR3 has the amino acid sequence of SEQ ID NO:88. In some embodiments, the antibody or antigen-binding fragment thereof comprises a heavy chain variable region that has at least 95% sequence identity to the amino acid sequence of SEQ ID NO:3, 5, 6, 7, 9, 10, 11 or 13, and a light chain variable region that has at least 95% sequence identity to the amino acid sequence of SEQ ID NO:4 or 12.

The present disclosure provides a fully human anti-CD38 antibody, or an antigen-binding fragment thereof, comprising a heavy chain variable region and a light chain variable region, the heavy chain variable region having at least 95% sequence identity to the amino acid sequence of SEQ ID NO:3, 5, 6, 7, 9, 10, 11 or 13; and the light chain variable region having at least 95% sequence identity to the amino acid sequence of SEQ ID NO:4 or 12.

The present disclosure provides a fully human anti-CD38 antibody, or an antigen-binding fragment thereof, comprising a heavy chain variable region and a light chain variable region, wherein the heavy chain variable region and the light chain variable region comprise the amino acid sequences of SEQ ID NOS:3 and 4, respectively (e.g., herein called 3H10m1); SEQ ID NOS:5 and 4, respectively (e.g., herein called 3G8m1); SEQ ID NOS:6 and 4, respectively (e.g., herein called 3E3m1); SEQ ID NOS:7 and 2, respectively (e.g., herein called 3G3); SEQ ID NOS:9 and 2, respectively (e.g., herein called 3E11); SEQ ID NOS:10 and 2, respectively (e.g., herein called 3H10); SEQ ID NOS:11 and 12, respectively (e.g., herein called 3H10N); SEQ ID NOS:13 and 12, respectively (e.g., herein called 3H10NS); SEQ ID NOS:1 and 4, respectively (e.g., herein called 3E10); or SEQ ID NOS:3 and 12, respectively (e.g., herein called 3H10m1g).

The present disclosure provides a fully human anti-CD38 antibody, or an antigen-binding fragment thereof, wherein the antigen-binding fragment is a Fab fragment comprising a variable domain region from a heavy chain and a variable domain region from a light chain, wherein (a) the variable domain region from the heavy chain comprises: a heavy chain complementarity determining region 1 (CDR1) having the amino acid sequence of SEQ ID NO:29, a heavy chain CDR2 having the amino acid sequence of SEQ ID NO:30, a heavy chain CDR3 having the amino acid sequence of SEQ ID NO:31; and the variable domain region from the light chain comprises: a light chain CDR1 having the amino acid sequence of SEQ ID NO:32, a light chain CDR2 having the amino acid sequence of SEQ ID NO:33, and a light chain CDR3 having the amino acid sequence of SEQ ID NO:34; wherein (b) the variable domain region from the heavy chain comprises: a heavy chain CDR1 having the amino acid sequence of SEQ ID NO:35, a heavy chain CDR2 having the amino acid sequence of SEQ ID NO:36, a heavy chain CDR3 having the amino acid sequence of SEQ ID NO:37; and the variable domain region from the light chain comprises: a light chain CDR1 having the amino acid sequence of SEQ ID NO:38, a light chain CDR2 having the amino acid sequence of SEQ ID NO:39, and a light chain CDR3 having the amino acid sequence of SEQ ID NO:40; wherein (c) the variable domain region from the heavy chain comprises: a heavy chain CDR1 having the amino acid sequence of SEQ ID NO:41, a heavy chain CDR2 having the amino acid sequence of SEQ ID NO:42, a heavy chain CDR3 having the amino acid sequence of SEQ ID NO:43; and the variable domain region from the light chain comprises: a light chain CDR1 having the amino acid sequence of SEQ ID NO:44, a light chain CDR2 having the amino acid sequence of SEQ ID NO:45, and a light chain CDR3 having the amino acid sequence of SEQ ID NO:46; wherein (d) the variable domain region from the heavy chain comprises: a heavy chain CDR1 having the amino acid sequence of SEQ ID NO:47, a heavy chain CDR2 having the amino acid sequence of SEQ ID NO:48, a heavy chain CDR3 having the amino acid sequence of SEQ ID NO:49; and the variable domain region from the light chain comprises: a light chain CDR1 having the amino acid sequence of SEQ ID NO:50, a light chain CDR2 having the amino acid sequence of SEQ ID NO:51, and a light chain CDR3 having the amino acid sequence of SEQ ID NO:52; wherein (e) the variable domain region from the heavy chain comprises: a heavy chain CDR1 having the amino acid sequence of SEQ ID NO:53, a heavy chain CDR2 having the amino acid sequence of SEQ ID NO:54, a heavy chain CDR3 having the amino acid sequence of SEQ ID NO:55; and the variable domain region from the light chain comprises: a light chain CDR1 having the amino acid sequence of SEQ ID NO:56, a light chain CDR2 having the amino acid sequence of SEQ ID NO:57, and a light chain CDR3 having the amino acid sequence of SEQ ID NO:58; wherein (f) the variable domain region from the heavy chain comprises: a heavy chain CDR1 having the amino acid sequence of SEQ ID NO:59, a heavy chain CDR2 having the amino acid sequence of SEQ ID NO:60, a heavy chain CDR3 having the amino acid sequence of SEQ ID NO:61; and the variable domain region from the light chain comprises: a light chain CDR1 having the amino acid sequence of SEQ ID NO:62, a light chain CDR2 having the amino acid sequence of SEQ ID NO:63, and a light chain CDR3 having the amino acid sequence of SEQ ID NO:64; wherein (g) the variable domain region from the heavy chain comprises: a heavy chain CDR1 having the amino acid sequence of SEQ ID NO:65, a heavy chain CDR2 having the amino acid sequence of SEQ ID NO:66, a heavy chain CDR3 having the amino acid sequence of SEQ ID NO:67; and the variable domain region from the light chain comprises: a light chain CDR1 having the amino acid sequence of SEQ ID NO:68, a light chain CDR2 having the amino acid sequence of SEQ ID NO:69, and a light chain CDR3 having the amino acid sequence of SEQ ID NO:70; wherein (h) the variable domain region from the heavy chain comprises: a heavy chain CDR1 having the amino acid sequence of SEQ ID NO:71, a heavy chain CDR2 having the amino acid sequence of SEQ ID NO:72, a heavy chain CDR3 having the amino acid sequence of SEQ ID NO:73; and the variable domain region from the light chain comprises: a light chain CDR1 having the amino acid sequence of SEQ ID NO:74, a light chain CDR2 having the amino acid sequence of SEQ ID NO:75, and a light chain CDR3 having the amino acid sequence of SEQ ID NO:76; wherein (i) the variable domain region from the heavy chain comprises: a heavy chain CDR1 having the amino acid sequence of SEQ ID NO:77, a heavy chain CDR2 having the amino acid sequence of SEQ ID NO:78, a heavy chain CDR3 having the amino acid sequence of SEQ ID NO:79; and the variable domain region from the light chain comprises: a light chain CDR1 having the amino acid sequence of SEQ ID NO:80, a light chain CDR2 having the amino acid sequence of SEQ ID NO:81, and a light chain CDR3 having the amino acid sequence of SEQ ID NO:82; or wherein (j) the variable domain region from the heavy chain comprises: a heavy chain CDR1 having the amino acid sequence of SEQ ID NO:83, a heavy chain CDR2 having the amino acid sequence of SEQ ID NO:84, a heavy chain CDR3 having the amino acid sequence of SEQ ID NO:85; and the variable domain region from the light chain comprises: a light chain CDR1 having the amino acid sequence of SEQ ID NO:86, a light chain CDR2 having the amino acid sequence of SEQ ID NO:87, and a light chain CDR3 having the amino acid sequence of SEQ ID NO:88.

The present disclosure provides a fully human anti-CD38 antibody, or an antigen-binding fragment thereof, wherein the antigen-binding fragment is a Fab fragment comprising a variable domain region from a heavy chain and a variable domain region from a light chain, wherein the variable domain region from the heavy chain comprises a sequence having at least 95% sequence identity to the amino acid sequence of SEQ ID NO:3, 5, 6, 7, 9, 10, 11 or 13, and wherein the variable domain region from the light chain comprises a sequence having at least 95% sequence identity to the amino acid sequence of SEQ ID NO:4 or 12.

The present disclosure provides a fully human anti-CD38 antibody, or an antigen-binding fragment thereof, wherein the antigen-binding fragment is a Fab fragment comprising a variable domain region from a heavy chain and a variable domain region from a light chain, wherein the variable domain region from the heavy chain and the variable domain region from the light chain are SEQ ID NOS:3 and 4, respectively (e.g., herein called 3H10m1); SEQ ID NOS:5 and 4, respectively (e.g., herein called 3G8m1); SEQ ID NOS:6 and 4, respectively (e.g., herein called 3E3m1); SEQ ID NOS:7 and 2, respectively (e.g., herein called 3G3); SEQ ID NOS:9 and 2, respectively (e.g., herein called 3E11); SEQ ID NOS:10 and 2, respectively (e.g., herein called 3H10); SEQ ID NOS:11 and 12, respectively (e.g., herein called 3H10N); SEQ ID NOS:13 and 12, respectively (e.g., herein called 3H10NS); SEQ ID NOS:1 and 4, respectively (e.g., herein called 3E10); or SEQ ID NOS:3 and 12, respectively (e.g., herein called 3H10m1g), optionally wherein the NGR motif at positions 54-56 of the heavy chain variable region is replaced with an SGR motif (see Table 1).

The present disclosure provides a fully human anti-CD38 antibody, or an antigen-binding fragment thereof, wherein the antigen-binding fragment is a single chain antibody comprising a variable domain region from a heavy chain and a variable domain region from a light chain joined together with a peptide linker, wherein (a) a heavy chain complementarity determining region 1 (CDR1) has the amino acid sequence of SEQ ID NO:29, a heavy chain CDR2 has the amino acid sequence of SEQ ID NO:30, a heavy chain CDR3 has the amino acid sequence of SEQ ID NO:31, a light chain CDR1 has the amino acid sequence of SEQ ID NO:32, a light chain CDR2 has the amino acid sequence of SEQ ID NO:33, and a light chain CDR3 has the amino acid sequence of SEQ ID NO:34 (e.g., herein called 3H10m1); (b) a heavy chain CDR1 has the amino acid sequence of SEQ ID NO:35, a heavy chain CDR2 has the amino acid sequence of SEQ ID NO:36, a heavy chain CDR3 has the amino acid sequence of SEQ ID NO:37, a light chain CDR1 has the amino acid sequence of SEQ ID NO:38, a light chain CDR2 has the amino acid sequence of SEQ ID NO:39, and a light chain CDR3 has the amino acid sequence of SEQ ID NO:40 (e.g., herein called 3G8m1); (c) a heavy chain CDR1 has the amino acid sequence of SEQ ID NO:41, a heavy chain CDR2 has the amino acid sequence of SEQ ID NO:42, a heavy chain CDR3 has the amino acid sequence of SEQ ID NO:43, a light chain CDR1 has the amino acid sequence of SEQ ID NO:44, a light chain CDR2 has the amino acid sequence of SEQ ID NO:45, and a light chain CDR3 has the amino acid sequence of SEQ ID NO:46 (e.g., herein called 3E3m1); (d) a heavy chain CDR1 has the amino acid sequence of SEQ ID NO:47, a heavy chain CDR2 has the amino acid sequence of SEQ ID NO:48, a heavy chain CDR3 has the amino acid sequence of SEQ ID NO:49, a light chain CDR1 has the amino acid sequence of SEQ ID NO:50, a light chain CDR2 has the amino acid sequence of SEQ ID NO:51, and a light chain CDR3 has the amino acid sequence of SEQ ID NO:52 (e.g., herein called 3G3); (e) a heavy chain CDR1 has the amino acid sequence of SEQ ID NO:53, a heavy chain CDR2 has the amino acid sequence of SEQ ID NO:54, a heavy chain CDR3 has the amino acid sequence of SEQ ID NO:55, a light chain CDR1 has the amino acid sequence of SEQ ID NO:56, a light chain CDR2 has the amino acid sequence of SEQ ID NO:57, and a light chain CDR3 has the amino acid sequence of SEQ ID NO:58 (e.g., herein called 3E11); (f) a heavy chain CDR1 has the amino acid sequence of SEQ ID NO:59, a heavy chain CDR2 has the amino acid sequence of SEQ ID NO:60, a heavy chain CDR3 has the amino acid sequence of SEQ ID NO:61, a light chain CDR1 has the amino acid sequence of SEQ ID NO:62, a light chain CDR2 has the amino acid sequence of SEQ ID NO:63, and a light chain CDR3 has the amino acid sequence of SEQ ID NO:64 (e.g., herein called 3H10); (g) a heavy chain CDR1 has the amino acid sequence of SEQ ID NO:65, a heavy chain CDR2 has the amino acid sequence of SEQ ID NO:66, a heavy chain CDR3 has the amino acid sequence of SEQ ID NO:67, a light chain CDR1 has the amino acid sequence of SEQ ID NO:68, a light chain CDR2 has the amino acid sequence of SEQ ID NO:69, and a light chain CDR3 has the amino acid sequence of SEQ ID NO:70 (e.g., herein called 3H10N); (h) a heavy chain CDR1 has the amino acid sequence of SEQ ID NO:71, a heavy chain CDR2 has the amino acid sequence of SEQ ID NO:72, a heavy chain CDR3 has the amino acid sequence of SEQ ID NO:73, a light chain CDR1 has the amino acid sequence of SEQ ID NO:74, a light chain CDR2 has the amino acid sequence of SEQ ID NO:75, and a light chain CDR3 has the amino acid sequence of SEQ ID NO:76 (e.g., herein called 3H10NS); (i) a heavy chain CDR1 has the amino acid sequence of SEQ ID NO:77, a heavy chain CDR2 has the amino acid sequence of SEQ ID NO:78, a heavy chain CDR3 has the amino acid sequence of SEQ ID NO:79, a light chain CDR1 has the amino acid sequence of SEQ ID NO:80, a light chain CDR2 has the amino acid sequence of SEQ ID NO:81, and a light chain CDR3 has the amino acid sequence of SEQ ID NO:82 (e.g., herein called 3E10); or (j) a heavy chain CDR1 has the amino acid sequence of SEQ ID NO:83, a heavy chain CDR2 has the amino acid sequence of SEQ ID NO:84, a heavy chain CDR3 has the amino acid sequence of SEQ ID NO:85, a light chain CDR1 has the amino acid sequence of SEQ ID NO:86, a light chain CDR2 has the amino acid sequence of SEQ ID NO:87, and a light chain CDR3 has the amino acid sequence of SEQ ID NO:88 (e.g., herein called 3H10m1g).

The present disclosure provides a fully human anti-CD38 antibody, or an antigen-binding fragment thereof, wherein the antigen-binding fragment is a single chain antibody comprising a variable domain region from a heavy chain and a variable domain region from a light chain joined together with a peptide linker, wherein the variable domain region from the heavy chain comprises a sequence having at least 95% sequence identity to the amino acid sequence of SEQ ID NO:3, 5, 6, 7, 9, 10, 11 or 13, and wherein the variable domain region from the light chain comprises a sequence having at least 95% sequence identity to the amino acid sequence of SEQ ID NO:4 or 12.

The present disclosure provides a fully human anti-CD38 antibody, or the antigen-binding fragment thereof, wherein the antigen-binding fragment is a single chain antibody comprising a variable domain region from a heavy chain and a variable domain region from a light chain joined together with a peptide linker, wherein the variable domain region from the heavy chain and the variable domain region from the light chain are SEQ ID NOS:3 and 4, respectively (e.g., herein called 3H10m1); SEQ ID NOS:5 and 4, respectively (e.g., herein called 3G8m1); SEQ ID NOS:6 and 4, respectively (e.g., herein called 3E3m1); SEQ ID NOS:7 and 2, respectively (e.g., herein called 3G3); SEQ ID NOS:9 and 2, respectively (e.g., herein called 3E11); SEQ ID NOS:10 and 2, respectively (e.g., herein called 3H10); SEQ ID NOS:11 and 12, respectively (e.g., herein called 3H10N); SEQ ID NOS:13 and 12, respectively (e.g., herein called 3H10NS); SEQ ID NOS:1 and 4, respectively (e.g., herein called 3E10); or SEQ ID NOS:3 and 12, respectively (e.g., herein called 3H10m1g), optionally wherein the NGR motif at positions 54-56 of the heavy chain variable region is replaced with an SGR motif (see Table 1).

In one embodiment, any of the fully human anti-CD38 antibodies disclosed herein, or any of the antigen-binding fragments thereof, is an IgG1, IgG2, IgG3 or IgG4 class antibody.

In one embodiment, any of the fully human anti-CD38 antibodies disclosed herein, or any of the antigen-binding fragments thereof, is an IgG1 or IgG4 class antibody.

In one embodiment, any of the fully human anti-CD38 antibodies disclosed herein, or any of the antigen-binding fragments thereof, bind to human CD38 protein (SEQ ID NO:19) and cross-reacts with CD38 protein from any one or any combination of two or more of CD38 proteins from cynomolgus (SEQ ID NO:20), mouse (SEQ ID NO:21) and/or rat (SEQ ID NO:22).

In one embodiment, any of the fully human anti-CD38 antibodies disclosed herein, or any of the antigen-binding fragments thereof, bind to human CD38 protein (SEQ ID NO:19) and do not cross-react with CD38 protein from cynomolgus (SEQ ID NO:20), mouse (SEQ ID NO:21) or rat (SEQ ID NO:22).

In one embodiment, any of the fully human anti-CD38 antibodies disclosed herein, or any of the antigen-binding fragments thereof, which bind human CD38 protein with a K_{D} of 10⁻⁸ M or less. In one embodiment, any of the fully human anti-CD38 antibodies disclosed herein, or any of the antigen-binding fragments thereof, bind cynomolgus CD38 protein with a K_{D} of 10⁻⁷ M or less. In one embodiment, any of the fully human anti-CD38 antibodies disclosed herein, or any of the antigen-binding fragments thereof, bind mouse CD38 protein with a K_{D} of 10⁻⁵ M or less.

In one embodiment, any of the fully human anti-CD38 antibodies disclosed herein, or any of the antigen-binding fragments thereof, bind to cells expressing or over-expressing CD38 protein including for example bind to human myeloma cells (e.g., human multiple myeloma cells), human B lymphoma cells, activated T cells, or cultured cell lines including RPMI8226, Raji or Ramos. In one embodiment, the cells expressing CD38 protein include transgenic cells engineered to express CD38 protein using recombinant DNA technology.

The present disclosure provides a pharmaceutical composition, comprising any one of the disclosed the human anti-CD38 antibodies, or any of the antigen-binding fragments thereof, and a pharmaceutically-acceptable excipient.

The present disclosure provides a kit, comprising any one of the disclosed the human anti-CD38 antibodies, or any of the antigen-binding fragments thereof.

The present disclosure provides one or more nucleic acids encoding an antigen-binding protein, antibody or antigen-binding fragment described herein. In some embodiments, the one or more nucleic acids are contained in one or more vectors. The one or more nucleic acids may be operably linked to one or more promoters. Also provided is a host cell comprising the one or more nucleic acids or vectors. Also provided are methods of producing an antigen-binding protein, antibody or antigen-binding fragment described herein, comprising culturing the host cell under conditions wherein the antigen-binding protein, antibody or antigen-binding fragment is produced.

The present disclosure provides a first nucleic acid that encodes a first polypeptide comprising an antibody heavy chain variable region having a heavy chain complementarity determining region (CDR) of any one of the disclosed the human anti-CD38 antibodies, including (a) a heavy chain complementarity determining region 1 (CDR1) having the amino acid sequence of SEQ ID NO:29, a heavy chain CDR2 having the amino acid sequence of SEQ ID NO:30, a heavy chain CDR3 having the amino acid sequence of SEQ ID NO:31; (b) a heavy chain CDR1 having the amino acid sequence of SEQ ID NO:35, a heavy chain CDR2 having the amino acid sequence of SEQ ID NO:36, a heavy chain CDR3 having the amino acid sequence of SEQ ID NO:37; (c) a heavy chain CDR1 having the amino acid sequence of SEQ ID NO:41, a heavy chain CDR2 having the amino acid sequence of SEQ ID NO:42, a heavy chain CDR3 having the amino acid sequence of SEQ ID NO:43; (d) a heavy chain CDR1 having the amino acid sequence of SEQ ID NO:47, a heavy chain CDR2 having the amino acid sequence of SEQ ID NO:48, a heavy chain CDR3 having the amino acid sequence of SEQ ID NO:49; (e) a heavy chain CDR1 having the amino acid sequence of SEQ ID NO:53, a heavy chain CDR2 having the amino acid sequence of SEQ ID NO:54, a heavy chain CDR3 having the amino acid sequence of SEQ ID NO:55; (f) a heavy chain CDR1 having the amino acid sequence of SEQ ID NO:59, a heavy chain CDR2 having the amino acid sequence of SEQ ID NO:60, a heavy chain CDR3 having the amino acid sequence of SEQ ID NO:61; (g) a heavy chain CDR1 having the amino acid sequence of SEQ ID NO:65, a heavy chain CDR2 having the amino acid sequence of SEQ ID NO:66, a heavy chain CDR3 having the amino acid sequence of SEQ ID NO:67; (h) a heavy chain CDR1 having the amino acid sequence of SEQ ID NO:71, a heavy chain CDR2 having the amino acid sequence of SEQ ID NO:72, a heavy chain CDR3 having the amino acid sequence of SEQ ID NO:73; (i) a heavy chain CDR1 having the amino acid sequence of SEQ ID NO:77, a heavy chain CDR2 having the amino acid sequence of SEQ ID NO:78, a heavy chain CDR3 having the amino acid sequence of SEQ ID NO:79; or (j) a heavy chain CDR1 having the amino acid sequence of SEQ ID NO:83, a heavy chain CDR2 having the amino acid sequence of SEQ ID NO:84, a heavy chain CDR3 having the amino acid sequence of SEQ ID NO:85.

The present disclosure provides a first vector comprising a first promoter operably linked to a first nucleic acid which encodes a first polypeptide comprising an antibody heavy chain variable region having the CDRs 1, 2 and 3, of any one of the disclosed the human anti-CD38 antibodies disclosed herein.

The present disclosure provides a first host cell harboring the first vector which comprises a first promoter operably linked to the first nucleic acid which encodes the first polypeptide comprising an antibody heavy chain variable region having the CDRs 1, 2 and 3, of any one of the disclosed the human anti-CD38 antibodies disclosed herein. In one embodiment, the first vector comprises a first expression vector. In one embodiment, the first host cell expresses the first polypeptide comprising the antibody heavy chain variable region having the CDRs 1, 2 and 3, of any one of the disclosed the human anti-CD38 antibodies disclosed herein.

The present disclosure provides a method for preparing a first polypeptide having an antibody heavy chain variable region comprising CDRs 1, 2 and 3, the method comprising: culturing a population (e.g., a plurality) of the first host cells harboring the first expression vector under conditions suitable for expressing the first polypeptide having the antibody heavy chain variable region comprising the CDRs 1, 2 and 3. In one embodiment, the method further comprises: recovering from the population of the first host cell the expressed first polypeptide having an antibody heavy chain variable region comprising the CDRs 1, 2 and 3.

The present disclosure provides a first nucleic acid that encodes a first polypeptide comprising an antibody heavy chain variable region having at least 95% sequence identity to the amino acid sequence of SEQ ID NO:3, 5, 6, 7, 9, 10, 11 or 13.

The present disclosure provides a first vector comprising a first promoter operably linked to the first nucleic acid which encodes the first polypeptide comprising the antibody heavy chain variable region having at least 95% sequence identity to the amino acid sequence of SEQ ID NO:3, 5, 6, 7, 9, 10, 11 or 13.

The present invention provides a first host cell harboring the first vector which comprises a first promoter operably linked to the first nucleic acid which encodes the first polypeptide comprising the antibody heavy chain variable region having at least 95% sequence identity to the amino acid sequence of SEQ ID NO:3, 5, 6, 7, 9, 10, 11 or 13. In one embodiment, the first vector comprises a first expression vector. In one embodiment, the first host cell expresses the first polypeptide comprising the antibody heavy chain variable region having at least 95% sequence identity to the amino acid sequence of SEQ ID NO:3, 5, 6, 7, 9, 10, 11 or 13.

The present disclosure provides a method for preparing a first polypeptide having an antibody heavy chain variable region, the method comprising: culturing a population (e.g., a plurality) of the first host cells harboring the first expression vector under conditions suitable for expressing the first polypeptide having the antibody heavy chain variable region having at least 95% sequence identity to the amino acid sequence of SEQ ID NO:3, 5, 6, 7, 9, 10, 11 or 13. In one embodiment, the method further comprises: recovering from the population of the first host cells the expressed first polypeptide having at least 95% sequence identity to the amino acid sequence of SEQ ID NO:3, 5, 6, 7, 9, 10, 11 or 13.

The present disclosure provides a second nucleic acid that encodes a polypeptide comprising an antibody light chain variable region having a light chain complementarity determining region (CDR) of any one of the disclosed the human anti-CD38 antibodies, including (a) a light chain CDR1 having the amino acid sequence of SEQ ID NO:32, a light chain CDR2 having the amino acid sequence of SEQ ID NO:33, and a light chain CDR3 having the amino acid sequence of SEQ ID NO:34; (b) a light chain CDR1 having the amino acid sequence of SEQ ID NO:38, a light chain CDR2 having the amino acid sequence of SEQ ID NO:39, and a light chain CDR3 having the amino acid sequence of SEQ ID NO:40; (c) a light chain CDR1 having the amino acid sequence of SEQ ID NO:44, a light chain CDR2 having the amino acid sequence of SEQ ID NO:45, and a light chain CDR3 having the amino acid sequence of SEQ ID NO:46; (d) a light chain CDR1 having the amino acid sequence of SEQ ID NO:50, a light chain CDR2 having the amino acid sequence of SEQ ID NO:51, and a light chain CDR3 having the amino acid sequence of SEQ ID NO:52; (e) a light chain CDR1 having the amino acid sequence of SEQ ID NO:56, a light chain CDR2 having the amino acid sequence of SEQ ID NO:57, and a light chain CDR3 having the amino acid sequence of SEQ ID NO:58; (f) a light chain CDR1 having the amino acid sequence of SEQ ID NO:62, a light chain CDR2 having the amino acid sequence of SEQ ID NO:63, and a light chain CDR3 having the amino acid sequence of SEQ ID NO:64; (g) a light chain CDR1 having the amino acid sequence of SEQ ID NO:68, a light chain CDR2 having the amino acid sequence of SEQ ID NO:69, and a light chain CDR3 having the amino acid sequence of SEQ ID NO:70; (h) a light chain CDR1 having the amino acid sequence of SEQ ID NO:74, a light chain CDR2 having the amino acid sequence of SEQ ID NO:75, and a light chain CDR3 having the amino acid sequence of SEQ ID NO:76; (i) a light chain CDR1 having the amino acid sequence of SEQ ID NO:80, a light chain CDR2 having the amino acid sequence of SEQ ID NO:81, and a light chain CDR3 having the amino acid sequence of SEQ ID NO:82; or (j) a light chain CDR1 having the amino acid sequence of SEQ ID NO:86, a light chain CDR2 having the amino acid sequence of SEQ ID NO:87, and a light chain CDR3 having the amino acid sequence of SEQ ID NO:88.

The present disclosure provides a second vector comprising a second promoter operably linked to a second nucleic acid which encodes a second polypeptide comprising an antibody light chain variable region having the CDRs 1, 2 and 3, of any one of the disclosed the human anti-CD38 antibodies disclosed herein.

The present disclosure provides a second host cell harboring the second vector which comprises a second promoter operably linked to the second nucleic acid which encodes the second polypeptide comprising an antibody light chain variable region having the CDRs 1, 2 and 3, of any one of the disclosed the human anti-CD38 antibodies disclosed herein. In one embodiment, the second vector comprises a second expression vector. In one embodiment, the second host cell expresses the second polypeptide comprising the antibody light chain variable region having the CDRs 1, 2 and 3, of any one of the disclosed the human anti-CD38 antibodies disclosed herein.

The present disclosure provides a method for preparing a second polypeptide having an antibody light chain variable region comprising CDRs 1, 2 and 3, the method comprising: culturing a population (e.g., a plurality) of the second host cells harboring the second expression vector under conditions suitable for expressing the second polypeptide having the antibody light chain variable region comprising the CDRs 1, 2 and 3. In one embodiment, the method further comprises: recovering from the population of the second host cell the expressed second polypeptide having an antibody light chain variable region comprising the CDRs 1, 2 and 3.

The present disclosure provides a second nucleic acid that encodes a second polypeptide comprising an antibody light chain variable region having at least 95% sequence identity to the amino acid sequence of SEQ ID NO:4 or 12.

The present disclosure provides a second vector comprising a second promoter operably linked to the second nucleic acid which encodes the second polypeptide comprising the antibody light chain variable region having at least 95% sequence identity to the amino acid sequence of SEQ ID NO:4 or 12.

The present invention provides a second host cell harboring the second vector which comprises a second promoter operably linked to the second nucleic acid which encodes the second polypeptide comprising the antibody light chain variable region having at least 95% sequence identity to the amino acid sequence of SEQ ID NO:4 or 12. In one embodiment, the second vector comprises a second expression vector. In one embodiment, the second host cell expresses the second polypeptide comprising the antibody light chain variable region having at least 95% sequence identity to the amino acid sequence of SEQ ID NO:4 or 12.

The present disclosure provides a method for preparing a second polypeptide having an antibody light chain variable region, the method comprising: culturing a population (e.g., a plurality) of the second host cells harboring the second expression vector under conditions suitable for expressing the second polypeptide having the antibody light chain variable region having at least 95% sequence identity to the amino acid sequence of SEQ ID NO:4 or 12. In one embodiment, the method further comprises: recovering from the population of the second host cells the expressed second polypeptide having at least 95% sequence identity to the amino acid sequence of SEQ ID NO:4 or 12.

The present disclosure provides a first nucleic acid that encodes a first polypeptide comprising an antibody heavy chain variable region having heavy chain complementarity determining regions (HC-CDRs 1, 2 and 3) of any one of disclosed the human anti-CD38 antibodies, and a second nucleic acid that encodes a second polypeptide comprising an antibody light chain variable region having light chain complementarity determining regions (LC-CDRs 1, 2 and 3) of any one of disclosed the human anti-CD38 antibodies, wherein (a) the heavy chain CDR 1, 2 and 3 regions comprise the amino acid sequences of SEQ ID NOS:29, 30 and 31, respectively, and the light chain CDR 1, 2 and 3 regions comprise the amino acid sequences of SEQ ID NOS:32, 33 and 34, respectively; or (b) the heavy chain CDR 1, 2 and 3 regions comprise the amino acid sequences of SEQ ID NOS:35, 36 and 37, respectively, and the light chain CDR 1, 2 and 3 regions comprise the amino acid sequences of SEQ ID NOS:38, 39 and 40, respectively; or (c) the heavy chain CDR 1, 2 and 3 regions comprise the amino acid sequences of SEQ ID NOS:41, 42 and 43, respectively, and the light chain CDR 1, 2 and 3 regions comprise the amino acid sequences of SEQ ID NOS:44, 45 and 46, respectively; or (d) the heavy chain CDR 1, 2 and 3 regions comprise the amino acid sequences of SEQ ID NOS:47, 48 and 49, respectively, and the light chain CDR 1, 2 and 3 regions comprise the amino acid sequences of SEQ ID NOS:50, 51 and 52, respectively; or (e) the heavy chain CDR 1, 2 and 3 regions comprise the amino acid sequences of SEQ ID NOS:53, 54 and 55, respectively, and the light chain CDR 1, 2 and 3 regions comprise the amino acid sequences of SEQ ID NOS:56, 57 and 58, respectively; or (f) the heavy chain CDR 1, 2 and 3 regions comprise the amino acid sequences of SEQ ID NOS:59, 60 and 61, respectively, and the light chain CDR 1, 2 and 3 regions comprise the amino acid sequences of SEQ ID NOS:62, 63 and 64, respectively; or (g) the heavy chain CDR 1, 2 and 3 regions comprise the amino acid sequences of SEQ ID NOS:65, 66 and 67, respectively, and the light chain CDR 1, 2 and 3 regions comprise the amino acid sequences of SEQ ID NOS:68, 69 and 70, respectively; or (h) the heavy chain CDR 1, 2 and 3 regions comprise the amino acid sequences of SEQ ID NOS:71, 72 and 73, respectively, and the light chain CDR 1, 2 and 3 regions comprise the amino acid sequences of SEQ ID NOS:74, 75 and 76, respectively; or (i) the heavy chain CDR 1, 2 and 3 regions comprise the amino acid sequences of SEQ ID NOS:77, 78 and 79, respectively, and the light chain CDR 1, 2 and 3 regions comprise the amino acid sequences of SEQ ID NOS:80, 81 and 82, respectively; or (j) the heavy chain CDR 1, 2 and 3 regions comprise the amino acid sequences of SEQ ID NOS:83, 84 and 85, respectively, and the light chain CDR 1, 2 and 3 regions comprise the amino acid sequences of SEQ ID NOS:86, 87 and 88, respectively.

The present disclosure provides a vector operably linked to the first nucleic acid that encodes the first polypeptide comprising an antibody heavy chain variable region having heavy chain complementarity determining regions (HC-CDRs 1, 2 and 3) of any one of disclosed the human anti-CD38 antibodies, and the vector is operably linked to the second nucleic acid that encodes the second polypeptide comprising the antibody light chain variable region having light chain complementarity determining regions (LC-CDRs 1, 2 and 3) of any one of disclosed the human anti-CD38 antibodies. In one embodiment, the vector comprises a promoter which is operably linked to the first and second nucleic acids. In one embodiment, the vector comprises a first promoter which is operably linked to the first nucleic acid, and the vector comprises a second promoter which is operably linked to the second nucleic acid.

The present disclosure provides a host cell harboring the vector which is operably linked to the first nucleic acid that encodes the first polypeptide comprising the heavy chain complementarity determining regions (HC-CDRs 1, 2 and 3) of any one of disclosed the human anti-CD38 antibodies and the vector comprises is operably linked to the second nucleic acid that encodes the second polypeptide comprising the light chain complementarity determining regions (LC-CDRs 1, 2 and 3) of any one of disclosed the human anti-CD38 antibodies. In one embodiment, the vector comprises an expression vector. In one embodiment, the vector comprises a promoter which is operably linked to the first and second nucleic acids. In one embodiment, the vector comprises a first promoter which is operably linked to the first nucleic acid, and the vector comprises a second promoter which is operably linked to the second nucleic acid. In one embodiment, the host cell expresses the first polypeptide comprising the heavy chain complementarity determining regions (HC-CDRs 1, 2 and 3) of any one of disclosed the human anti-CD38 antibodies and the second polypeptide comprising the light chain complementarity determining regions (LC-CDRs 1, 2 and 3) of any one of disclosed the human anti-CD38 antibodies.

The present disclosure provides a method for preparing the first and second polypeptides, the method comprising: culturing a population (e.g., a plurality) of the host cell harboring the expression vector under conditions suitable for expressing the first polypeptide comprising the heavy chain complementarity determining regions (HC-CDRs 1, 2 and 3) of any one of disclosed the human anti-CD38 antibodies and the second polypeptide comprising the light chain complementarity determining regions (LC-CDRs 1, 2 and 3) of any one of disclosed the human anti-CD38 antibodies. In one embodiment, the method further comprises: recovering from the population of the host cells the expressed first and second polypeptides.

The present disclosure provides a first nucleic acid that encodes a first polypeptide comprising an antibody heavy chain variable region having at least 95% sequence identity to the amino acid sequence of SEQ ID NO:3, 5, 6, 7, 9, 10, 11 or 13, and a second nucleic acid that encodes a second polypeptide comprising an antibody light chain variable region having at least 95% sequence identity to the amino acid sequence of SEQ ID NO:4 or 12.

The present disclosure provides a vector operably linked to the first nucleic acid that encodes a first polypeptide comprising an antibody heavy chain variable region having at least 95% sequence identity to the amino acid sequence of SEQ ID NO:3, 5, 6, 7, 9, 10, 11 or 13, and the vector is operably linked to the second nucleic acid which encodes the second polypeptide comprising the antibody light chain variable region having at least 95% sequence identity to the amino acid sequence of SEQ ID NO:4 or 12. In one embodiment, the vector comprises a promoter which is operably linked to the first and second nucleic acids. In one embodiment, the vector comprises a first promoter which is operably linked to the first nucleic acid, and the vector comprises a second promoter which is operably linked to the second nucleic acid.

The present invention provides a host cell harboring the vector which is operably linked to the first nucleic acid that encodes a first polypeptide comprising an antibody heavy chain variable region having at least 95% sequence identity to the amino acid sequence of SEQ ID NO:3, 5, 6, 7, 9, 10, 11 or 13, and the vector is operably linked to the second nucleic acid which encodes the second polypeptide comprising the antibody light chain variable region having at least 95% sequence identity to the amino acid sequence of SEQ ID NO:4 or 12. In one embodiment, the vector comprises an expression vector. In one embodiment, the vector comprises a promoter which is operably linked to the first and second nucleic acids. In one embodiment, the vector comprises a first promoter which is operably linked to the first nucleic acid, and the vector comprises a second promoter which is operably linked to the second nucleic acid. In one embodiment, the host cell expresses the first polypeptide comprising an antibody heavy chain variable region having at least 95% sequence identity to the amino acid sequence of SEQ ID NO:3, 5, 6, 7, 9, 10, 11 or 13, and the host cell expresses the second polypeptide comprising the antibody light chain variable region having at least 95% sequence identity to the amino acid sequence of SEQ ID NO:4 or 12.

The present disclosure provides a method for preparing a first polypeptide having an antibody heavy chain variable region and a second polypeptide having an antibody light chain variable region, the method comprising: culturing a population (e.g., a plurality) of the host cells harboring the expression vector under conditions suitable for expressing the first polypeptide comprising an antibody heavy chain variable region having at least 95% sequence identity to the amino acid sequence of SEQ ID NO:3, 5, 6, 7, 9, 10, 11 or 13, and for expressing the second polypeptide having the antibody light chain variable region having at least 95% sequence identity to the amino acid sequence of SEQ ID NO:4 or 12. In one embodiment, the method further comprises: recovering from the population of the host cells the expressed first polypeptide comprising an antibody heavy chain variable region having at least 95% sequence identity to the amino acid sequence of SEQ ID NO:3, 5, 6, 7, 9, 10, 11 or 13, and the expressed second polypeptide having at least 95% sequence identity to the amino acid sequence of SEQ ID NO:4 or 12.

The present disclosure provides a first nucleic acid that encodes a first polypeptide comprising an antibody heavy chain variable region having heavy chain complementarity determining regions (HC-CDRs 1, 2 and 3) of any one of disclosed the human anti-CD38 antibodies, and a second nucleic acid that encodes a second polypeptide comprising an antibody light chain variable region having light chain complementarity determining regions (LC-CDRs 1, 2 and 3) of any one of disclosed the human anti-CD38 antibodies, wherein (a) the heavy chain CDR 1, 2 and 3 regions comprise the amino acid sequences of SEQ ID NOS:29, 30 and 31, respectively, and the light chain CDR 1, 2 and 3 regions comprise the amino acid sequences of SEQ ID NOS:32, 33 and 34, respectively; or (b) the heavy chain CDR 1, 2 and 3 regions comprise the amino acid sequences of SEQ ID NOS:35, 36 and 37, respectively, and the light chain CDR 1, 2 and 3 regions comprise the amino acid sequences of SEQ ID NOS:38, 39 and 40, respectively; or (c) the heavy chain CDR 1, 2 and 3 regions comprise the amino acid sequences of SEQ ID NOS:41, 42 and 43, respectively, and the light chain CDR 1, 2 and 3 regions comprise the amino acid sequences of SEQ ID NOS:44, 45 and 46, respectively; or (d) the heavy chain CDR 1, 2 and 3 regions comprise the amino acid sequences of SEQ ID NOS:47, 48 and 49, respectively, and the light chain CDR 1, 2 and 3 regions comprise the amino acid sequences of SEQ ID NOS:50, 51 and 52, respectively; or (e) the heavy chain CDR 1, 2 and 3 regions comprise the amino acid sequences of SEQ ID NOS:53, 54 and 55, respectively, and the light chain CDR 1, 2 and 3 regions comprise the amino acid sequences of SEQ ID NOS:56, 57 and 58, respectively; or (f) the heavy chain CDR 1, 2 and 3 regions comprise the amino acid sequences of SEQ ID NOS:59, 60 and 61, respectively, and the light chain CDR 1, 2 and 3 regions comprise the amino acid sequences of SEQ ID NOS:62, 63 and 64, respectively; or (g) the heavy chain CDR 1, 2 and 3 regions comprise the amino acid sequences of SEQ ID NOS:65, 66 and 67, respectively, and the light chain CDR 1, 2 and 3 regions comprise the amino acid sequences of SEQ ID NOS:68, 69 and 70, respectively; or (h) the heavy chain CDR 1, 2 and 3 regions comprise the amino acid sequences of SEQ ID NOS:71, 72 and 73, respectively, and the light chain CDR 1, 2 and 3 regions comprise the amino acid sequences of SEQ ID NOS:74, 75 and 76, respectively; or (i) the heavy chain CDR 1, 2 and 3 regions comprise the amino acid sequences of SEQ ID NOS:77, 78 and 79, respectively, and the light chain CDR 1, 2 and 3 regions comprise the amino acid sequences of SEQ ID NOS:80, 81 and 82, respectively; or (j) the heavy chain CDR 1, 2 and 3 regions comprise the amino acid sequences of SEQ ID NOS:83, 84 and 85, respectively, and the light chain CDR 1, 2 and 3 regions comprise the amino acid sequences of SEQ ID NOS:86, 87 and 88, respectively.

The present disclosure provides a first vector comprising a first promoter operably linked to the first nucleic acid that encodes the first polypeptide comprising an antibody heavy chain variable region having heavy chain complementarity determining regions (HC-CDRs 1, 2 and 3) of any one of disclosed the human anti-CD38 antibodies, and a second vector comprising a second promoter operably linked to the second nucleic acid that encodes the second polypeptide comprising the antibody light chain variable region having light chain complementarity determining regions (LC-CDRs 1, 2 and 3) of any one of disclosed the human anti-CD38 antibodies.

The present disclosure provides a host cell harboring the first vector which comprises the first promoter operably linked to the first nucleic acid that encodes the first polypeptide comprising the heavy chain complementarity determining regions (HC-CDRs 1, 2 and 3) of any one of disclosed the human anti-CD38 antibodies, and the host cell harbors the second vector which comprises a second promoter operably linked to the second nucleic acid that encodes the second polypeptide comprising the light chain complementarity determining regions (LC-CDRs 1, 2 and 3) of any one of disclosed the human anti-CD38 antibodies. In one embodiment, the first and second vectors are first and second expression vectors, respectively. In one embodiment, the host cell expresses the first polypeptide comprising the heavy chain complementarity determining regions (HC-CDRs 1, 2 and 3) of any one of disclosed the human anti-CD38 antibodies and the host cell expresses the second polypeptide comprising the light chain complementarity determining regions (LC-CDRs 1, 2 and 3) of any one of disclosed the human anti-CD38 antibodies.

The present disclosure provides a method for preparing the first and second polypeptides, the method comprising: culturing a population (e.g., a plurality) of the host cell harboring the first and second expression vectors under conditions suitable for expressing the first polypeptide comprising the heavy chain complementarity determining regions (HC-CDRs 1, 2 and 3) of any one of disclosed the human anti-CD38 antibodies and the second polypeptide comprising the light chain complementarity determining regions (LC-CDRs 1, 2 and 3) of any one of disclosed the human anti-CD38 antibodies. In one embodiment, the method further comprises: recovering from the population of the host cells the expressed first and second polypeptides.

The present disclosure provides a first nucleic acid that encodes a first polypeptide comprising an antibody heavy chain variable region having at least 95% sequence identity to the amino acid sequence of SEQ ID NO:3, 5, 6, 7, 9, 10, 11 or 13, and a second nucleic acid that encodes a second polypeptide comprising an antibody light chain variable region having at least 95% sequence identity to the amino acid sequence of SEQ ID NO:4 or 12.

The present disclosure provides a first vector comprising a first promoter operably linked to the first nucleic acid that encodes a first polypeptide comprising an antibody heavy chain variable region having at least 95% sequence identity to the amino acid sequence of SEQ ID NO:3, 5, 6, 7, 9, 10, 11 or 13, and a second vector comprising a second promoter operably linked to the second nucleic acid which encodes the second polypeptide comprising the antibody light chain variable region having at least 95% sequence identity to the amino acid sequence of SEQ ID NO:4 or 12.

The present invention provides a host cell harboring the first vector which comprises a first promoter operably linked to the first nucleic acid that encodes a first polypeptide comprising an antibody heavy chain variable region having at least 95% sequence identity to the amino acid sequence of SEQ ID NO:3, 5, 6, 7, 9, 10, 11 or 13, and the host cell harbors the second vector which comprises a second promoter operably linked to the second nucleic acid which encodes the second polypeptide comprising the antibody light chain variable region having at least 95% sequence identity to the amino acid sequence of SEQ ID NO:4 or 12. In one embodiment, the first and second vectors comprise a first and second expression vector, respectively. In one embodiment, the host cell expresses the first polypeptide comprising an antibody heavy chain variable region having at least 95% sequence identity to the amino acid sequence of SEQ ID NO:3, 5, 6, 7, 9, 10, 11 or 13, and the host cell expresses the second polypeptide comprising the antibody light chain variable region having at least 95% sequence identity to the amino acid sequence of SEQ ID NO:4 or 12.

The present disclosure provides a method for preparing a first polypeptide having an antibody heavy chain variable region and a second polypeptide having an antibody light chain variable region, the method comprising: culturing a population (e.g., a plurality) of the host cells harboring the first and second expression vectors under conditions suitable for expressing the first polypeptide comprising an antibody heavy chain variable region having at least 95% sequence identity to the amino acid sequence of SEQ ID NO:3, 5, 6, 7, 9, 10, 11 or 13, and for expressing the second polypeptide having the antibody light chain variable region having at least 95% sequence identity to the amino acid sequence of SEQ ID NO:4 or 12. In one embodiment, the method further comprises: recovering from the population of the host cells the expressed first polypeptide comprising an antibody heavy chain variable region having at least 95% sequence identity to the amino acid sequence of SEQ ID NO:3, 5, 6, 7, 9, 10, 11 or 13, and the expressed second polypeptide having at least 95% sequence identity to the amino acid sequence of SEQ ID NO:4 or 12.

The present disclosure provides a nucleic acid that encodes a polypeptide (e.g., a single chain antibody including an scFv) comprising an antibody heavy chain variable region having heavy chain complementarity determining regions (HC-CDRs 1, 2 and 3) of any one of disclosed the human anti-CD38 antibodies, and an antibody light chain variable region having light chain complementarity determining regions (LC-CDRs 1, 2 and 3) of any one of disclosed the human anti-CD38 antibodies, wherein (a) the heavy chain CDR 1, 2 and 3 regions comprise the amino acid sequences of SEQ ID NOS:29, 30 and 31, respectively, and the light chain CDR 1, 2 and 3 regions comprise the amino acid sequences of SEQ ID NOS:32, 33 and 34, respectively; or (b) the heavy chain CDR 1, 2 and 3 regions comprise the amino acid sequences of SEQ ID NOS:35, 36 and 37, respectively, and the light chain CDR 1, 2 and 3 regions comprise the amino acid sequences of SEQ ID NOS:38, 39 and 40, respectively; or (c) the heavy chain CDR 1, 2 and 3 regions comprise the amino acid sequences of SEQ ID NOS:41, 42 and 43, respectively, and the light chain CDR 1, 2 and 3 regions comprise the amino acid sequences of SEQ ID NOS:44, 45 and 46, respectively; or (d) the heavy chain CDR 1, 2 and 3 regions comprise the amino acid sequences of SEQ ID NOS:47, 48 and 49, respectively, and the light chain CDR 1, 2 and 3 regions comprise the amino acid sequences of SEQ ID NOS:50, 51 and 52, respectively; or (e) the heavy chain CDR 1, 2 and 3 regions comprise the amino acid sequences of SEQ ID NOS:53, 54 and 55, respectively, and the light chain CDR 1, 2 and 3 regions comprise the amino acid sequences of SEQ ID NOS:56, 57 and 58, respectively; or (f) the heavy chain CDR 1, 2 and 3 regions comprise the amino acid sequences of SEQ ID NOS:59, 60 and 61, respectively, and the light chain CDR 1, 2 and 3 regions comprise the amino acid sequences of SEQ ID NOS:62, 63 and 64, respectively; or (g) the heavy chain CDR 1, 2 and 3 regions comprise the amino acid sequences of SEQ ID NOS:65, 66 and 67, respectively, and the light chain CDR 1, 2 and 3 regions comprise the amino acid sequences of SEQ ID NOS:68, 69 and 70, respectively; or (h) the heavy chain CDR 1, 2 and 3 regions comprise the amino acid sequences of SEQ ID NOS:71, 72 and 73, respectively, and the light chain CDR 1, 2 and 3 regions comprise the amino acid sequences of SEQ ID NOS:74, 75 and 76, respectively; or (i) the heavy chain CDR 1, 2 and 3 regions comprise the amino acid sequences of SEQ ID NOS:77, 78 and 79, respectively, and the light chain CDR 1, 2 and 3 regions comprise the amino acid sequences of SEQ ID NOS:80, 81 and 82, respectively; or (j) the heavy chain CDR 1, 2 and 3 regions comprise the amino acid sequences of SEQ ID NOS:83, 84 and 85, respectively, and the light chain CDR 1, 2 and 3 regions comprise the amino acid sequences of SEQ ID NOS:86, 87 and 88, respectively.

The present disclosure provides a vector comprising a promoter operably linked to the nucleic acid that encodes the polypeptide (e.g., a single chain antibody including an scFv) comprising the antibody heavy chain variable region having heavy chain complementarity determining regions (HC-CDRs 1, 2 and 3) of any one of disclosed the human anti-CD38 antibodies, and the antibody light chain variable region having light chain complementarity determining regions (LC-CDRs 1, 2 and 3) of any one of disclosed the human anti-CD38 antibodies.

The present disclosure provides a host cell harboring the vector which comprises a promoter operably linked to the nucleic acid that encodes the polypeptide (e.g., a single chain antibody including an scFv) comprising the antibody heavy chain variable region having heavy chain complementarity determining regions (HC-CDRs 1, 2 and 3) of any one of disclosed the human anti-CD38 antibodies, and the antibody light chain variable region having light chain complementarity determining regions (LC-CDRs 1, 2 and 3) of any one of disclosed the human anti-CD38 antibodies. In one embodiment, the vector comprises an expression vector. In one embodiment, the host cell expresses the polypeptide comprising the antibody heavy chain variable region having heavy chain complementarity determining regions (HC-CDRs 1, 2 and 3) of any one of disclosed the human anti-CD38 antibodies, and the antibody light chain variable region having light chain complementarity determining regions (LC-CDRs 1, 2 and 3) of any one of disclosed the human anti-CD38 antibodies.

The present disclosure provides a method for preparing a polypeptide described herein, the method comprising: culturing a population (e.g., a plurality) of a host cell harboring an expression vector described herein under conditions suitable for expressing the polypeptide. In some embodiments, the polypeptide comprises the antibody heavy chain variable region having heavy chain complementarity determining regions (HC-CDRs 1, 2 and 3) of any one of disclosed the human anti-CD38 antibodies, and the antibody light chain variable region having light chain complementarity determining regions (LC-CDRs 1, 2 and 3) of any one of disclosed the human anti-CD38 antibodies. In one embodiment, the method further comprises: recovering from the population of the host cells the expressed polypeptide.

The present disclosure provides a nucleic acid that encodes a polypeptide (e.g., a single chain antibody including an scFv) comprising an antibody heavy chain variable region having at least 95% sequence identity to the amino acid sequence of SEQ ID NO:3, 5, 6, 7, 9, 10, 11 or 13, and a an antibody light chain variable region having at least 95% sequence identity to the amino acid sequence of SEQ ID NO:4 or 12.

The present disclosure provides a vector comprising a promoter operably linked to the nucleic acid that encodes the polypeptide (e.g., a single chain antibody including an scFv) comprising the antibody heavy chain variable region having at least 95% sequence identity to the amino acid sequence of SEQ ID NO:3, 5, 6, 7, 9, 10, 11 or 13, and the antibody light chain variable region having at least 95% sequence identity to the amino acid sequence of SEQ ID NO:4 or 12.

The present invention provides a host cell harboring the vector which comprises a promoter operably linked to the nucleic acid that encodes the polypeptide (e.g., a single chain antibody including an scFv) comprising the antibody heavy chain variable region having at least 95% sequence identity to the amino acid sequence of SEQ ID NO:3, 5, 6, 7, 9, 10, 11 or 13, and the antibody light chain variable region having at least 95% sequence identity to the amino acid sequence of SEQ ID NO:4 or 12. In one embodiment, the vector comprises an expression vector. In one embodiment, the host cell expresses the polypeptide (e.g., a single chain antibody including an scFv) comprising the antibody heavy chain variable region having at least 95% sequence identity to the amino acid sequence of SEQ ID NO:3, 5, 6, 7, 9, 10, 11 or 13, and the antibody light chain variable region having at least 95% sequence identity to the amino acid sequence of SEQ ID NO:4 or 12.

The present disclosure provides a method for preparing the polypeptide (e.g., a single chain antibody including an scFv), the method comprising: culturing a population (e.g., a plurality) of the host cells harboring the expression vector under conditions suitable for expressing the polypeptide (e.g., a single chain antibody including an scFv) comprising the antibody heavy chain variable region having at least 95% sequence identity to the amino acid sequence of SEQ ID NO:3, 5, 6, 7, 9, 10, 11 or 13, and the antibody light chain variable region having at least 95% sequence identity to the amino acid sequence of SEQ ID NO:4 or 12. In one embodiment, the method further comprises: recovering from the population of the host cells the expressed the polypeptide (e.g., a single chain antibody including an scFv) comprising the antibody heavy chain variable region having at least 95% sequence identity to the amino acid sequence of SEQ ID NO:3, 5, 6, 7, 9, 10, 11 or 13, and the antibody light chain variable region having at least 95% sequence identity to the amino acid sequence of SEQ ID NO:4 or 12.

The present disclosure provides a method (e.g., an in vitro method) for inhibiting growth or proliferation of CD38-expressing cells, comprising: contacting (i) a population (e.g., a plurality) of effector cells with (ii) a population (e.g., a plurality) of target cells which express CD38 (iii) in the presence of any one or any combination of 2-3 of the human anti-CD38 antibodies described herein, under conditions that are suitable for inhibiting growth or proliferation of the CD38-expressing cells. In one embodiment, the population of effector cells comprises PBMCs or NK cells. In one embodiment, the population of target cells comprise multiple myeloma (MM) cells expressing CD38 or transgenic cells expressing CD38. In one embodiment, the ratio of the effector-to-target cells is 1:1, 2:1, 3:1, 4:1 or 5:1. In one embodiment, the ratio of the effector-to-target cells is 5-10:1, 10-20:1, or 20-30:1.

The present disclosure provides a method (e.g., an in vitro method) for killing CD38-expressing cells, comprising: contacting (i) a population (e.g., a plurality) of effector cells with (ii) a population (e.g., a plurality) of target cells which express CD38 (iii) in the presence of any one or any combination of 2-3 of the human anti-CD38 antibodies described herein, under conditions that are suitable for killing the CD38-expressing cells. In one embodiment, the population of effector cells comprises PBMCs or NK cells. In one embodiment, the population of target cells comprise a multiple myeloma (MM) cells expressing CD38 or transgenic cells expressing CD38. In one embodiment, the ratio of the effector-to-target cells is 1:1, 2:1, 3:1, 4:1 or 5:1. In one embodiment, the ratio of the effector-to-target cells is 5-10:1, 10-20:1, or 20-30:1.

The present disclosure provides a method for treating a subject having a disease associated with CD38 over-expression, the method comprising: administering to the subject an effective amount of a therapeutic composition comprising any one or any combination of 2-3 of the human anti-CD38 antibodies described herein. In one embodiment, the disease associated with CD38 over-expression comprises: a B-cell leukemia, B-cell lymphoma or B-cell myeloma. In one embodiment, the disease associated with CD38 over-expression is selected from a group consisting of multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CIVIL), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris; scleroderma, multiple sclerosis, anti-phospholipid syndrome, ANCA associated vasculitis Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

### DESCRIPTION OF THE DRAWINGS

FIG. 1 shows an SPR sensorgram of binding kinetics of an anti-CD38 antibody (Daratumumab).

FIG. 2 shows an SPR sensorgram of binding kinetics of an anti-CD38 antibody (parent antibody A2).

FIG. 3 shows an SPR sensorgram of binding kinetics of an anti-CD38 antibody (variant antibody 3H10m1).

FIG. 4 shows an SPR sensorgram of binding kinetics of an anti-CD38 antibody (variant antibody 3G3).

FIG. 5 shows an SPR sensorgram of binding kinetics of an anti-CD38 antibody (variant antibody 3E10).

FIG. 6 shows a table that summarizes binding kinetics obtained from SPR data of parent antibody A2, and variant antibodies 3E10 and 3H10m1, compared to Daratumumab.

FIG. 7 shows histograms of flow cytometry data of non-activated T cells stained with various anti-CD38 antibodies including parent antibody A2, variant antibodies 3H10m1 and 3E1, and Daratumumab.

FIG. 8A shows histograms of flow cytometry data of activated T cells stained with various anti-CD38 antibodies including parent antibody A2, variant antibodies 3H10m1 and 3E1, and Daratumumab.

FIG. 8B shows flow cytometry data of activated T cells stained with anti-CD47 H3D4 antibody.

FIG. 9 shows a bar graph of a binding titration assay of RPMI 8226 cells stained with various anti-CD38 antibodies including parent antibody A2, variant antibodies 3H10m1 and 3G3 and 3E1, and Daratumumab. Each data set includes three different antibody concentrations (from left to right) of 10 ug/mL, 1 ug/mL and 0.1 ug/mL.

FIG. 10 shows a graph of a cell binding assay of various anti-CD38 antibodies binding to human B lymphoma cell line Raji, including anti-CD38 scFv-Fc (line A), parent antibody A2 (line B), variant antibody 3G3 (line C), variant antibody 3H10m1 (line D) and Daratumumab (line E).

FIG. 11 shows a graph of a cell binding assay of various anti-CD38 antibodies binding to human B lymphoma cell line Ramos, including anti-CD38 scFv-Fc (line A), parent antibody A2 (line B), Daratumumab (line C), variant antibody 3G3 (line D) and variant antibody 3H10m1 (line E).

FIG. 12 shows a graph of a cell binding assay of various anti-CD38 antibodies binding to human primary T cells, including anti-CD38 scFv-Fc (line A), parent antibody A2 (line B), variant antibody 3G3 (line C), Daratumumab (line D), and variant antibody 3H10m1 (line E).

FIG. 13 shows a bar graph of a species cross-reactivity assay of Daratumumab compared to anti-CD38-A2 parent antibody.

FIG. 14 shows flow cytometry data of a species cross-reactivity assay of parent antibody A2 compared to variant antibodies 3G3 and 3H10m1, and Daratumumab.

FIG. 15 shows a bar graph of an antibody-dependent cellular phagocytosis (ADCP) assay comparing killing activity of A2 parent antibody, with variant antibodies 3G3 and 3H10m1, and Daratumumab.

FIG. 16 shows a graph of an antibody-dependent cellular cytotoxicity (ADCC) assay comparing killing activity of various anti-CD38 antibodies, including parent antibody A2 (line A), Daratumumab (line B), anti-CD38 scFv-Fc (line C), variant antibody 3H10m1 (line D) and variant antibody 3G3 (line E).

FIG. 17 shows an SPR sensorgram of ranked affinities of variant antibodies 3G3, 3H10, 3H10m1, 3E11, 3E3m1 and 3G8m1.

### DEFINITIONS

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to cleavage, for example cleavage by a secretory signal peptide or by non-enzymatic cleavage at certain amino acid residues. Polypeptides include mature molecules that have undergone cleavage. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. Polypeptides comprising amino acid sequences of binding proteins that bind CD38 (e.g., anti-CD38 variant antibodies or variant antigen-binding portions thereof) prepared using recombinant procedures are described herein.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof. In one embodiment, nucleic acids comprise a one type of polynucleotides or a mixture of two or more different types of polynucleotides. Nucleic acids encoding the antibody variant light chains, antibody variant heavy chains, anti-CD38 variant antibodies or variant antigen-binding portions thereof, are described herein. In one embodiment, nucleic acids encode a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13. In one embodiment, nucleic acids encode a light chain variable region comprising at least 95% sequence identity to the amino acid sequence of SEQ ID NO:4 or SEQ ID NO:12.

The term “recover” or “recovery” or “recovering”, and other related terms, refers to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide sequence which mediates secretion of the expressed protein. The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., an antibody or an antigen binding portion thereof) or polynucleotide that is substantially free of naturally associated components (e.g., other cellular material, or components associated with cellular expression system). A protein may be rendered substantially free of naturally associated components (or components associated with a cellular expression system or chemical synthesis methods used to produce the antibody) by isolation, using protein purification techniques well known in the art. The term isolated also refers in some embodiments to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrophotometry. In one embodiment, any of the antibody variant light chains, antibody variant heavy chains, anti-CD38 variant antibodies or variant antigen binding protein described herein can be isolated.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins that bind CD38 are described herein.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. Antigen binding proteins having immunoglobulin-like properties that bind specifically to CD38 are described herein.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)2 fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Anti-CD38 variant antibodies, comprising variant light and/or heavy chains are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains. Antigen binding domains from anti-CD38 variant antibodies are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵M or less, or 10⁻⁶ M or less, or 10⁻⁷M or less, or 10⁻⁸M or less, or 10⁻⁹M or less, or 10⁻¹⁰ M or less. Anti-CD38 variant antibodies that specifically bind CD38 are described herein.

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. Anti-CD38 variant antibodies, and variant antigen binding proteins thereof, that bind an epitope of a CD38 polypeptide (antigen) are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. Antigen-binding fragments of anti-CD38 variant antibodies are described herein.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises Vu and Cm regions. An Fv fragment comprises V_{L} and V_{H} regions. An Fv can bind an antigen. A dAb fragment has a V_{H} domain, a V_{L} domain, or an antigen-binding fragment of a V_{H} or V_{L} domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989). Fab fragments comprising antigen binding portions from anti-CD38 variant antibodies are described herein.

A single-chain antibody (scFv) is an antibody in which a V_{L} and a V_{H} region are joined via a linker (e.g., a synthetic sequence of amino acid residues) to form a continuous protein chain. Preferably the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83). Single chain antibodies comprising antigen binding portions from anti-CD38 variant antibodies are described herein.

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the anti-CD38 variant antibodies described herein.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Fully human anti-CD38 antibodies and antigen binding proteins thereof that are variant antibodies are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Chimeric antibodies can be prepared from portions of any of the anti-CD38 variant antibodies described herein.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

The term “Fc” or “Fc region” as used herein refers to the portion of an antibody heavy chain constant region beginning in or after the hinge region and ending at the C-terminus of the heavy chain. The Fc region comprises at least a portion of the CH and CH3 regions and may, or may not, include a portion of the hinge region. Two polypeptide chains each carrying a half Fc region can dimerize to form a full Fc domain. An Fc domain can bind Fc cell surface receptors and some proteins of the immune complement system. An Fc domain exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc domain can bind an Fc receptor, including FcγRI (e.g., CD64), FcγRII (e.g, CD32) and/or FcγRIII (e.g., CD16a).

The term “labeled antibody” or related terms as used herein refers to antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the anti-CD38 variant antibodies described herein can be unlabeled or can be joined to a detectable label or moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test antibody may be similar but not identical to any of the amino acid sequences of the light chain and/or heavy chain polypeptides that make up any of the anti-CD38 variant antibodies, or variant antigen binding protein thereof, described herein. The similarities between the test antibody and the polypeptides can be at least 95%, or at or at least 96% identical, or at least 97% identical, or at least 98% identical, or at least 99% identical, to any of the light chain and/or heavy chain polypeptides that make up any of the anti-CD38 variant antibodies, or variant antigen binding protein thereof, described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. A skilled artisan can introduce up to 5% conservative and/or non-conservative amino acid substitutions in a heavy chain variable region and/or light chain variable region without negatively impacting the physical structure, binding capability or cell killing capability of an antibody. Well known methods for identifying and making conservative amino acid substitutions in a variable region that are designed to retain or improve antibody binding characteristics are described in: Brummel, et al., 1993 Biochemistry 32:1180-1187; Kobayashi et al., 1999 Protein Engineering 12(10):879-884; and Burks et al., 1997 Proc. Natl. Acad. Sci. USA 94:412-417). Methods for identifying and making non-conservative amino acid substitutions in a heavy chain and/or light chain variable region to retain or improve antigen binding are also known (Near et al., 1993 Molecular Immunology 30(4):369-377). Thus, a skilled artisan can predict and change up to 5% of the amino acids in a heavy chain variable region and/or light chain variable region without significantly diminishing antigen binding capability of an antibody. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise nucleic acids that encode at least a portion of any of the light chain, heavy chain or anti-CD38 variant antibodies described herein.

Vectors (e.g., expression vectors) operably linked to a nucleic acid encoding the antibody variant light chains, antibody variant heavy chains, anti-CD38 variant antibodies or variant antigen-binding portions thereof, are described herein. In one embodiment, a vector is operably linked a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13. In one embodiment, nucleic acids encode a light chain variable region comprising at least 95% sequence identity to the amino acid sequence of SEQ ID NO:4 or SEQ ID NO:12.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been transfected, transformed or transduced with exogenous nucleic acid (transgene). The host cell includes the primary subject cell and its progeny. Exogenous nucleic acids encoding at least a portion of any of the light chain, heavy chain or anti-CD38 variant antibodies described herein can be introduced into a host cell. Expression vectors comprising at least a portion of any of the light chain, heavy chain or anti-CD38 variant antibodies described herein can be introduced into a host cell, and the host cell can express polypeptides comprising at least a portion of the light chain, heavy chain or anti-CD38 variant antibody.

The terms “host cell” or “or a population of host cells” or related terms as used herein refer to a cell (or a population thereof) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. Progeny cells may or may not harbor identical genetic material compared to the parent cell. Host cells encompass progeny cells. In one embodiment, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host's genome or can harbor an extrachromosomal expression vector. In one embodiment, host cells and populations thereof can harbor an extrachromosomal vector that is present after several cell divisions or is present transiently and is lost after several cell divisions.

Transgenic host cells can be prepared using non-viral methods, including well-known designer nucleases including zinc finger nucleases, TALENS or CRISPR/Cas. A transgene can be introduced into a host cell's genome using genome editing technologies such as zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host's genome and the endonuclease domain makes a double-stranded cut. The donor DNA carries the transgene, for example any of the nucleic acids encoding a CAR or DAR construct described herein, and flanking sequences that are homologous to the regions on either side of the intended insertion site in the host cell's genome. The host cell's DNA repair machinery enables precise insertion of the transgene by homologous DNA repair. Transgenic mammalian host cells have been prepared using zinc finger nucleases (U.S. Pat. Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host's DNA. Transgenic host cells can be prepared using CRISPR (Clustered Regularly Interspaced Short Palindromic Repeats). CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Examples of CRISPR/Cas systems used to modify genomes are described for example in U.S. Pat. Nos. 8,697,359, 10,000,772, 9,790,490, and U. S. Patent Application Publication No. US 2018/0346927. In one embodiment, transgenic host cells can be prepared using zinc finger nuclease, TALEN or CRISPR/Cas system, and the host target site can be a TRAC gene (T Cell Receptor Alpha Constant). The donor DNA can include for example any of the nucleic acids encoding a CAR or DAR construct described herein. Electroporation, nucleofection or lipofection can be used to co-deliver into the host cell the donor DNA with the zinc finger nuclease, TALEN or CRISPR/Cas system.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In one embodiment, host cells comprise non-human cells including CHO, BHK, NS0, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

A host cell, or a population of host cells, harboring a vector (e.g., an expression vector) operably linked to a nucleic acid encoding the antibody variant light chains, antibody variant heavy chains, anti-CD38 variant antibodies or variant antigen-binding portions thereof, are described herein. In one embodiment, a host cell harbors a vector operably linked a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13. In one embodiment, nucleic acids encode a light chain variable region comprising at least 95% sequence identity to the amino acid sequence of SEQ ID NO:4 or SEQ ID NO:12.

Polypeptides of the present disclosure (e.g., antibodies and antigen binding proteins) can be produced using any methods known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts. Any of the light chain, heavy chain or anti-CD38 variant antibodies, or variant antigen binding protein thereof, can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are preferably at least 65% pure, at least 75% pure, at least 85% pure, more preferably at least 95% pure, and most preferably at least 98% pure. Regardless of the exact numerical value of the purity, the polypeptide is sufficiently pure for use as a pharmaceutical product. Any of the light chain, heavy chain or anti-CD38 variant antibodies, or variant antigen binding protein thereof, described herein can be expressed by transgenic host cells and then purified to about 65-98% purity or high level of purity using any art-known method.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. A preferred form of glycosylation is sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

In one embodiment, the antibodies and antigen binding proteins described herein can be modified to become soluble polypeptides which comprises linking the Antibodies and antigen binding proteins to non-proteinaceous polymers. In one embodiment, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337.

PEG is a water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ—CH₂CH₂OH (1), where n is 20 to 2300 and X is H or a terminal modification, e.g., a C₁₋₄ alkyl. In one embodiment, the PEG terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. One form of PEGs includes two PEG side-chains (PEG2) linked via the primary amino groups of a lysine (Monfardini et al., Bioconjugate Chem. 6 (1995) 62-69).

The serum clearance rate of PEG-modified polypeptide may be modulated (e.g., increased or decreased) by about 10%, 20%, 30%, 40%, 50%, 60%, 70%, 80%, or even 90%, relative to the clearance rate of the unmodified antibodies and antigen binding proteins binding polypeptides. The PEG-modified antibodies and antigen binding proteins may have a half-life (t_{1/2}) which is enhanced relative to the half-life of the unmodified polypeptide. The half-life of PEG-modified polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified antibodies and antigen binding proteins. In some embodiments, the protein half-life is determined in vitro, such as in a buffered saline solution or in serum. In other embodiments, the protein half-life is an in vivo half-life, such as the half-life of the protein in the serum or other bodily fluid of an animal.

The present disclosure provides therapeutic compositions comprising any of the light chain, heavy chain or anti-CD38 variant antibodies, or variant antigen binding protein thereof, described herein in and a pharmaceutically-acceptable excipient. An excipient encompasses carriers, stabilizers and excipients. Excipients of pharmaceutically acceptable excipients includes for example inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the anti-CD38 variant antibodies (or variant antigen binding portions thereof) may be administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the antibody (or antigen binding portions thereof) is formulated in the presence of sodium acetate to increase thermal stability.

Any of the variant anti-CD38 antibodies (or variant antigen binding portions thereof) may be formulated for oral use include tablets containing the active ingredient(s) in a mixture with non-toxic pharmaceutically acceptable excipients. Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the anti-CD38 variant antibodies described herein (or variant antigen binding protein thereof) can be administered to a subject using art-known methods and delivery routes.

The terms “effective amount”, “therapeutically effective amount” or “effective dose” or related terms may be used interchangeably and refer to an amount of antibody or an antigen binding protein (e.g., any of the anti-CD38 variant antibodies described herein or variant antigen binding protein thereof) that when administered to a subject, is sufficient to effect a measurable improvement or prevention of a disease or disorder associated with tumor or cancer antigen expression. Therapeutically effective amounts of antibodies provided herein, when used alone or in combination, will vary depending upon the relative activity of the antibodies and combinations (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered at about 0.01 g/kg to about 50 mg/kg per day, preferably 0.01 mg/kg to about 30 mg/kg per day, most preferably 0.1 mg/kg to about 20 mg/kg per day. The polypeptide may be administered daily (e.g., once, twice, three times, or four times daily) or preferably less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

The present disclosure provides methods for treating a subject having a disease associated with expression of CD38. The disease comprises cancer or tumor cells expressing the tumor-associated antigens. In one embodiment, the cancer or tumor includes cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the cancer comprises hematological cancers, including leukemias, lymphomas, myelomas and B cell lymphomas. Hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis. Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

An anti-CD38 parent antibody is disclosed in U.S. patent application publication No. US 2016/0297888 A1, published 13 Oct. 2016 (the disclosure of which is incorporated by reference herein in its entirety), granted as U.S. Pat. No. 10,059,774 on Aug. 28, 2018. This antibody, referred to herein as the “parent” and/or “wild type” antibody, comprises a parent heavy chain having a heavy variable region comprising the amino acid sequence of SEQ ID NO:1, and a parent light chain having a light variable region comprising the amino acid sequence of SEQ ID NO:2. The parent antibody disclosed herein is designated “A2”.

The present disclosure provides anti-CD38 antigen-binding proteins, including anti-CD38 variant antibodies, or antigen-binding portions thereof, that specifically bind CD38 and uses thereof. The anti-CD38 variant antibodies can exhibit improved characteristics compared to the parent antibody A2, where the improved characteristics include improved binding to CD38 antigen, improved binding to CD38-expressing cells and/or higher levels of cytotoxicity. The anti-CD38 variant antibodies, like parent antibody A2, can cross-react (bind) with cynomolgus CD38 antigen.

In some embodiments, the present disclosure provides an antigen-binding protein, such as a fully human antibody of an IgG class, that binds to an epitope of a CD38 polypeptide (e.g., target antigen) or fragment of a CD38 polypeptide, wherein the antibody is a variant antibody having a heavy chain variable region and/or light chain variable region that differs from the parent antibody A2. In one embodiment, the CD38 target antigen comprises a naturally-occurring polypeptide (e.g., UniProtKB accession number P28907 (NP 0017766.2)) having a wild-type or polymorphic or mutant amino acid sequence. The CD38 target antigen can be prepared by recombinant methods or can be chemically synthesized. The CD38 target antigen can be in soluble form or membrane-bound form (e.g., expressed by a cell or phage). In one embodiment, the CD38 target antigen comprises an extracellular portion of a cell surface CD38 antigen. In one embodiment, the CD38 target antigen is expressed by a cell, for example a cancer or non-cancer cell line that naturally expresses CD38 such as Raji, Ramos, Daudi, MOLT-4, Karpas-707, REH, U-266/70, U-698, RPMI-8226, A549, or expressed by a cell line that is engineered to express CD38 such as CHO, HeLa, HEK293 or Panoply™ (from Creative Biogene, Shirley, N.Y.). Cell lines that do not naturally express CD38 are not expected to bind an anti-CD38 antibody, such as for example K562, A-431, ARH-77, PC-3 and HEK 293. The CD38 target antigen can be a fusion protein or conjugated for example with a detectable moiety such as a fluorophore. The CD38 target antigen can be a recombinant polypeptide with or without a histidine-tag. The CD38 target antigen can be a CD38 his-tagged protein from human, mouse or cynomolgus (e.g., from Sino Biological, catalog #10818-H08H, 50191-M08H, or 90050-C08H, respectively). In one embodiment, the CD38 polypeptide comprises the amino acid sequence of SEQ ID NO:19.

In one embodiment, wild type and/or mutated human CD38 antigen can be used in an assay comparing binding capabilities of any of the anti-CD38 variant antibodies described herein compared to the anti-CD38 parent antibody (A2), and/or in an epitope mapping assay comparing binding capabilities of any of the anti-CD38 variant antibodies described herein compared to the anti-CD38 parent antibody (A2).

In some embodiments, the present disclosure provides an antigen-binding protein, such as a fully human antibody of an IgG class, that binds to an epitope of a CD38 polypeptide (target antigen), wherein the antibody is a variant antibody comprising a heavy chain variable region having at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the anti-CD38 variant antibody comprises a light chain variable region having 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof; or wherein the antibody is a variant antibody comprising a heavy chain variable region having at least 95% sequence identity, or at least 96% sequence identity, or at least 97% sequence identity. In some embodiments, the present disclosure provides an antigen-binding protein, such as a fully human antibody of an IgG class, that binds to an epitope of a CD38 polypeptide (target antigen), wherein the antibody is a variant antibody comprising a heavy chain variable region having at least 95% sequence identity, or at least 96% sequence identity, or at least 97% sequence identity, or at least 98% sequence identity, or at least 99% sequence identity to the amino acid sequence of at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the anti-CD38 variant antibody comprises a light chain variable region having 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof. In one embodiment, the anti-CD38 variant antibody is an isolated antibody. In one embodiment, the anti-CD38 variant antibody is a recombinant antibody. In one embodiment, the anti-CD38 variant antibody comprises an IgG1, IgG2, IgG3 or IgG4 class antibody. In one embodiment, the anti-CD38 variant antibody comprises an IgG1 or IgG4 class antibody. In one embodiment, the hinge region of an anti-CD38 antibody can be mutated to alter the number of potential disulfide bond formation. In one embodiment, the anti-CD38 variant antibody comprises a hinge region having the amino acid sequence CPPC, CPSC, SPPC or SPSC. In one embodiment, the anti-CD38 variant antibody comprises a heavy chain constant region having a hinge region wherein the amino acid sequence CPSC, SPPC or SPSC replaces the sequence CPPC (e.g., see bold and underlined sequence at positions 109-112 of SEQ ID NO:14, 15 or 16). In one embodiment, the heavy chain of an anti-CD38 antibody can be mutated to eliminate one or more NG motifs (e.g., as part of an NGR motif) that are known to isomerize. In one embodiment, the isomerized site can bind integrin. In one embodiment, the anti-CD38 variant antibody comprises a heavy chain that includes an SGR motif that replaces an NGR motif. In one embodiment, the anti-CD38 variant antibody comprises a heavy chain variable region wherein an SGR motif replaces an NGR motif (e.g., see bold and underlined sequence at positions 54-56 of SEQ ID NO:3, 5, 6, 7, 9, 10, 11 or 13 in Table 1). In one embodiment, the anti-CD38 variant antibody comprises a light chain constant region comprising the amino acid sequence of SEQ ID NO:17 or 18. In one embodiment, the heavy and/or light chain of an anti-CD38 antibody can be mutated, and the mutated antibody exhibits the same or similar binding capabilities to CD38 antigen and/or CD38-expressing cells.

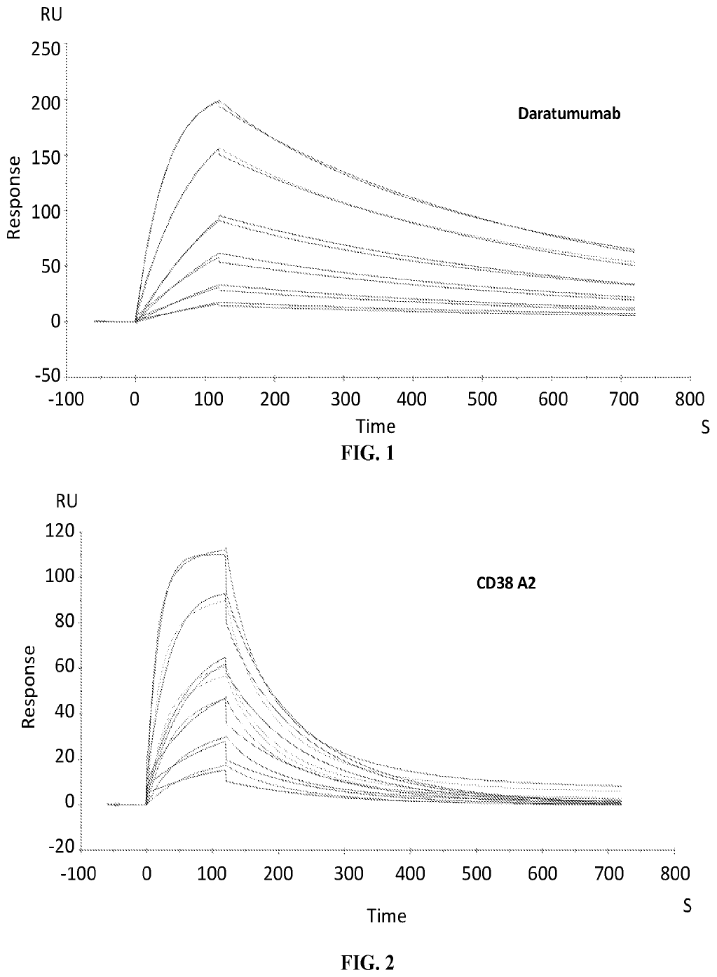

In one embodiment, the anti-CD38 variant antibody, or fragment thereof, comprises an antigen binding portion that binds an epitope of a CD38 polypeptide (target antigen) with a binding affinity (K_{D}) of 10⁻⁶ M or less, 10⁻⁷ M or less, 10⁻⁸ M or less, 10⁻⁹M or less, or 10⁻¹⁰ M or less (see FIGS. 1-6). In one embodiment, binding between the anti-CD38 variant antibody, or fragment thereof, can be detected and measured using surface plasmon resonance, flow cytometry and/or ELISA.

The present disclosure provides an anti-CD38 variant antibody which binds an epitope of a CD38 polypeptide from a human, and can bind (e.g., cross-react) with an epitope of a CD38 polypeptide (e.g., homologous antigen) from at least one of a non-human animal such as mouse, rat, goat, rabbit, hamster and/or monkey (e.g., cynomolgus). In one embodiment, the anti-CD38 variant antibody binds mouse CD38 with a binding affinity K_{D} of 10⁻⁵M or less, or 10⁻⁶ M or less, or 10⁻⁷M or less, or 10⁻⁸M or less, or 10⁻⁹M or less, or 10⁻¹⁰ M or less. In one embodiment, the anti-CD38 variant antibody binds cynomolgus CD38 with a binding affinity K_{D} of 10⁻⁵M or less, or 10⁻⁶ M or less, or 10⁻⁷ M or less, or 10⁻⁸M or less, or 10⁻⁹ M or less, or 10⁻¹⁰ M or less. In one embodiment, the non-human CD38 comprises a mouse CD38 polypeptide (e.g., from Sino Biological, catalog #50191-M08H), or a cynomolgus CD38 polypeptide (e.g, from Sino Biological, catalog #90050-C08H).

The present disclosure provides a fully human antibody that binds a CD38 polypeptide, wherein the antibody is a variant antibody comprising both heavy and light chains, wherein the heavy/light chain variable region amino acid sequences have at least 95% sequence identity, or at least 96% sequence identity, or at least 97% sequence identity, or at least 98% sequence identity, or at least 99% sequence identity to any of the following amino acid sequence sets: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein).

The present disclosure provides a Fab fully human antibody fragment which is a variant antibody fragment comprising a variable region from a heavy chain and a variable region from a light chain. In some embodiments, the sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof. In some embodiments, the sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides a Fab fully human antibody fragment which is a variant antibody fragment comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein).

The present disclosure provides a single chain fully human antibody which is a variant single chain antibody comprising a polypeptide chain having a variable region from a fully human heavy chain and a variable region from a fully human light chain, and optionally a linker (e.g., peptide linker) joining the variable heavy and variable light chain regions. In some embodiments, the variable heavy region comprises at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the variable light region comprises at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof. In some embodiments, the variable heavy region comprises at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the variable light region comprises at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure provides a single chain fully human antibody which is a variant single chain antibody comprising a polypeptide chain having heavy chain variable region and a light chain variable region, wherein the heavy/light chain variable region amino acid sequence sets are at least 95% identical, or at least 96% identical, or at least 97% identical, or at least 98% identical, or at least 99% identical to any of the following amino acid sequence sets: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein). In one embodiment, the single chain fully human antibody comprises an optional linker (e.g., peptide linker) joining the variable heavy and variable light chain regions.

The present disclosure provides therapeutic compositions comprising any of the anti-CD38 variant antibodies described herein, or variant antigen binding proteins thereof, and a pharmaceutically-acceptable excipient. An excipient encompasses carriers and stabilizers. In one embodiment, the therapeutic compositions comprise an anti-CD38 variant antibody, or variant antigen binding fragment thereof, comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein).

The present disclosure provides nucleic acids encoding an antibody heavy chain variable region having at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof. The present disclosure provides nucleic acids encoding an antibody light chain variable region having at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure provides nucleic acids encoding an antibody heavy chain variable region having at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof. The present disclosure provides nucleic acids encoding an antibody light chain variable region having at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure provides nucleic acids encoding an antibody heavy chain variable region having at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the nucleic acids encode an antibody light chain variable region having at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure provides nucleic acids encoding an antibody heavy chain variable region having at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the nucleic acids encode an antibody light chain variable region having at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure provides nucleic acids encoding a variant antibody comprising both heavy and light chains, wherein the heavy/light chain variable region amino acid sequences have at least 95% sequence identity, or at least 96% sequence identity, or at least 97% sequence identity, or at least 98% sequence identity, or at least 99% sequence identity to any of the following amino acid sequence sets: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein).

The present disclosure provides nucleic acids encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof. The present disclosure provides nucleic acids encoding a Fab fully human antibody fragment which is a variant antibody fragment comprising a variable region from a heavy chain and a variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides nucleic acids encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof. The present disclosure provides nucleic acids encoding a Fab fully human antibody fragment which is a variant antibody fragment comprising a variable region from a heavy chain and a variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides nucleic acids encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the nucleic acids encode the variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides nucleic acids encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the nucleic acids encode the variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides nucleic acids encoding a Fab fully human antibody fragment which is a variant antibody fragment comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein).

The present disclosure provides nucleic acids encoding a single chain fully human antibody comprising a polypeptide chain having a variable region from a fully human heavy chain and a variable region from a fully human light chain, and optionally a linker (e.g., peptide linker) joining the variable heavy and variable light chain regions. In some embodiments, the variable heavy region comprises at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the variable light region which comprises at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure provides nucleic acids encoding a single chain fully human antibody comprising a polypeptide chain having a variable region from a fully human heavy chain and a variable region from a fully human light chain, and optionally a linker (e.g., peptide linker) joining the variable heavy and variable light chain regions. In some embodiments, the variable heavy region comprises at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the variable light region which comprises at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure provides nucleic acids encoding a single chain fully human antibody which is a variant single chain antibody comprising a polypeptide chain having heavy chain variable region and a light chain variable region, wherein the heavy/light chain variable region amino acid sequence sets are at least 95% identical, or at least 96% identical, or at least 97% identical, or at least 98% identical, or at least 99% identical to any of the following amino acid sequence sets: SEQ ID NOS: SEQ ID NOS: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein). In one embodiment, the nucleic acid encodes a single chain fully human antibody comprising an optional linker (e.g., peptide linker) joining the variable heavy and variable light chain regions.

The present disclosure provides a first vector (e.g., a first expression vector) operably linked to a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, and a second vector (e.g., second expression vector) operably linked to a nucleic acid encoding a light chain variable region comprising at least 95% sequence identity to the amino acid sequence of SEQ ID NO:4 or SEQ ID NO:12. The present disclosure also provides a first vector (e.g., a first expression vector) operably linked to a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, and a second vector (e.g., second expression vector) operably linked to a nucleic acid encoding a light chain variable region comprising at least 95% sequence identity to the amino acid sequence of SEQ ID NO:4 or SEQ ID NO:12.

The present disclosure provides a first vector (e.g., a first expression vector) operably linked to a nucleic acid encoding a heavy chain variable region comprising at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13. In one embodiment, the first vector is also operably linked to a nucleic acid encoding a light chain variable region comprising at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12. The present disclosure also provides a first vector (e.g., a first expression vector) operably linked to a nucleic acid encoding a heavy chain variable region comprising at least 95% sequence identity, or at least 96% sequence identity, or at least 97% sequence identity, or at least 98% sequence identity, or at least 99% sequence identity to the amino acid sequence of SEQ ID NO: 1, SEQ ID NO:3, SEQ ID NO:5, SEQ ID NO:6, SEQ ID NO:7, SEQ ID NO:9, SEQ ID NO:10, SEQ ID NO:11 or SEQ ID NO:13, which is also operably linked to a nucleic acid encoding a light chain variable region comprising at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12.

The present disclosure provides a vector (e.g., an expression vector) encoding a nucleic acid encoding a variant antibody comprising both heavy and light chains, wherein the heavy/light chain variable region amino acid sequences have at least 95% sequence identity, or at least 96% sequence identity, or at least 97% sequence identity, or at least 98% sequence identity, or at least 99% sequence identity to any of the following amino acid sequence sets: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein).

The present disclosure provides a first vector (e.g., a first expression vector) operably linked to a nucleic acid encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof. The present disclosure also a second vector (e.g., a second expression vector) operably linked to a nucleic acid encoding a Fab fully human antibody fragment which is a variant antibody fragment comprising a variable region from a heavy chain and a variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides a first vector (e.g., a first expression vector) operably linked to a nucleic acid encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof. The present disclosure also a second vector (e.g., a second expression vector) operably linked to a nucleic acid encoding a Fab fully human antibody fragment which is a variant antibody fragment comprising a variable region from a heavy chain and a variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides a vector (e.g., an expression vector) operably linked to a nucleic acid encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the vector is operably linked to nucleic acids encoding the variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides a vector (e.g., an expression vector) operably linked to a nucleic acid encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the vector is operably linked to nucleic acids encoding the variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof.

The present disclosure provides a vector (e.g., an expression vector) operably linked to nucleic acids encoding a Fab fully human antibody fragment which is a variant antibody fragment comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein).

The present disclosure provides a first vector (e.g., a first expression vector) operably linked to a nucleic acid encoding a single chain fully human antibody comprising a polypeptide chain having a variable region from a fully human heavy chain and a variable region from a fully human light chain, and optionally a linker (e.g., peptide linker) joining the variable heavy and variable light chain regions, wherein the variable heavy region comprises at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and wherein the variable light region comprises at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure also provides a first vector (e.g., a first expression vector) operably linked to a nucleic acid encoding a single chain fully human antibody comprising a polypeptide chain having a variable region from a fully human heavy chain and a variable region from a fully human light chain, and optionally a linker (e.g., peptide linker) joining the variable heavy and variable light chain regions, wherein the variable heavy region comprises at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and wherein the variable light region comprises at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof.

The present disclosure provides a host cell, or a population of host cells, wherein the host cell or individual host cells from the population of host cells harbors a first vector (e.g., a first expression vector) operably linked to a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, and the host cell or individual host cells from the population of host cells harbors a second vector (e.g., a second expression vector) operably linked to a nucleic acid encoding a light chain variable region comprising at least 95% sequence identity to the amino acid sequence of SEQ ID NO:4 or SEQ ID NO:12. In one embodiment, the first expression vector directs expression of the heavy chain variable region and the second expression vector directs expression of the light chain variable region in the host cell or the population of host cells.

The present disclosure also provides a host cell, or a population of host cells, wherein the host cell or individual host cells from the population of host cells harbors a first vector (e.g., a first expression vector) operably linked to a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, and the host cell or individual host cells from the population of host cells harbors a second vector (e.g., a second expression vector) operably linked to a nucleic acid encoding a light chain variable region comprising at least 95% sequence identity to the amino acid sequence of SEQ ID NO:4 or SEQ ID NO:12. In one embodiment, the first expression vector directs expression of the heavy chain variable region and the second expression vector directs expression of the light chain variable region in the host cell or the population of host cells.

The present disclosure provides a first host cell, or a first population of host cells, wherein the first host cell or individual host cells from the first population of host cells harbors a first vector (e.g., a first expression vector) operably linked to a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, and a second host cell or a second population of host cells, wherein the second host cell or individual host cells from the second population of host cells harbors a second vector (e.g., a first expression vector) operably linked to a nucleic acid encoding a light chain variable region comprising at least 95% sequence identity to the amino acid sequence of SEQ ID NO:4 or SEQ ID NO:12. In one embodiment, the first expression vector directs expression of the heavy chain variable region in the first host cell, and the second expression vector directs expression of the light chain variable region in the second host cell.

The present disclosure also provides a first host cell, or a first population of host cells, wherein the first host cell or individual host cells from the first population of host cells harbors a first vector (e.g., a first expression vector) operably linked to a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, and a second host cell or a second population of host cells, wherein the second host cell or individual host cells from the second population of host cells harbors a second vector (e.g., a first expression vector) operably linked to a nucleic acid encoding a light chain variable region comprising at least 95% sequence identity to the amino acid sequence of SEQ ID NO:4 or SEQ ID NO:12. In one embodiment, the first expression vector directs expression of the heavy chain variable region in the first host cell, and the second expression vector directs expression of the light chain variable region in the second host cell.

The present disclosure provides a host cell, or a population of host cells, wherein the host cell or individual host cells from the population of host cells harbors a vector (e.g., an expression vector) operably linked to a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, and the vector in the host cell is also operably linked to a nucleic acid encoding a light chain variable region comprising at least 95% sequence identity to the amino acid sequence of SEQ ID NO:4 or SEQ ID NO:12. In one embodiment, the expression vector directs expression of the heavy chain variable region and the light chain variable region in the host cell.

The present disclosure also provides a host cell, or a population of host cells, wherein the host cell or individual host cells from the population of host cells harbors a vector (e.g., an expression vector) operably linked to a nucleic acid encoding a heavy chain variable region comprising at least 95% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, and the vector in the host cell is also operably linked to a nucleic acid encoding a light chain variable region comprising at least 95% sequence identity to the amino acid sequence of SEQ ID NO:4 or SEQ ID NO:12. In one embodiment, the expression vector directs expression of the heavy chain variable region and the light chain variable region in the host cell.

The present disclosure provides a host cell, or a population of host cells, wherein the host cell or individual host cells from the population of host cells harbors a first vector (e.g., a first expression vector) operably linked to a nucleic acid encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof. In one embodiment, the host cell or individual host cells from the population of host cells harbors also harbors a second vector (e.g., a second expression vector) operably linked to a nucleic acid encoding a Fab fully human antibody fragment which is a variant antibody fragment comprising a variable region from a heavy chain and a variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof. In one embodiment, the first expression vector directs expression of the heavy chain variable region and the second expression vector directs expression of the light chain variable region in the host cell.

The present disclosure provides a host cell, or a population of host cells, wherein the host cell or individual host cells from the population of host cells harbors a first vector (e.g., a first expression vector) operably linked to a nucleic acid encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof. In one embodiment, the host cell or individual host cells from the population of host cells harbors also harbors a second vector (e.g., a second expression vector) operably linked to a nucleic acid encoding a Fab fully human antibody fragment which is a variant antibody fragment comprising a variable region from a heavy chain and a variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof. In one embodiment, the first expression vector directs expression of the heavy chain variable region and the second expression vector directs expression of the light chain variable region in the host cell.

The present disclosure provides a host cell, or a population of host cells, wherein the host cell or individual host cells from the population of host cells harbors a first vector (e.g., a first expression vector) operably linked to a nucleic acid encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the first vector is operably linked to nucleic acids encoding the variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof. In one embodiment, the first expression vector directs expression of the heavy chain variable region and the light chain variable region in the host cell.

The present disclosure provides a host cell, or a population of host cells, wherein the host cell or individual host cells from the population of host cells harbors a first vector (e.g., a first expression vector) operably linked to a nucleic acid encoding a Fab fully human antibody fragment which is a variant antibody fragment comprising a variable region from a heavy chain and a variable region from a light chain, wherein the amino acid sequence of the variable region from the heavy chain is at least 95% identical, or at least 96% identical, or at least 97% identical, or at least 98% identical, or at least 99% identical to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and the first vector is operably linked to nucleic acids encoding the variable region from a light chain, wherein the amino acid sequence of the variable region from the light chain is at least 95% identical, or at least 96% identical, or at least 97% identical, or at least 98% identical, or at least 99% identical to the amino acid sequence of SEQ ID NO:4 or SEQ ID NO:12, or combinations thereof. In one embodiment, the first expression vector directs expression of the heavy chain variable region and the light chain variable region in the host cell.

The present disclosure provides a host cell, or a population of host cells, wherein the host cell or individual host cells from the population of host cells harbors a vector (e.g., an expression vector) operably linked to nucleic acids encoding a Fab fully human antibody fragment which is a variant antibody fragment comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein). In one embodiment, the expression vector directs expression of the heavy/light chain variable regions in the host cell.

The present disclosure provides a host cell, or a population of host cells, wherein the host cell or individual host cells from the population of host cells harbors a first vector (e.g., a first expression vector) operably linked to a nucleic acid encoding any of the following amino acid sequence sets: SEQ ID NOS:3 and 4 (called 3H10m1 herein), SEQ ID NOS:5 and 4 (called 3G8m1 herein), SEQ ID NOS:6 and 4 (called 3E3m1 herein), SEQ ID NOS:7 and 2 (called 3G3 herein), SEQ ID NOS:9 and 2 (called 3E11 herein), SEQ ID NOS:10 and 2 (called 3H10 herein), SEQ ID NOS:11 and 12 (called 3H10N herein), SEQ ID NOS:13 and 12 (called 3H10NS herein), SEQ ID NOS:1 and 4 (called 3E10 herein) or SEQ ID NOS:3 and 12 (called 3H10m1g herein). In one embodiment, the expression vector directs expression of the heavy/light chain variable regions in the host cell.

The present disclosure provides a host cell, or a population of host cells, wherein the host cell or individual host cells from the population of host cells harbors a first vector (e.g., a first expression vector) operably linked to a nucleic acid encoding a single chain fully human antibody comprising a polypeptide chain having a variable region from a fully human heavy chain and a variable region from a fully human light chain, and optionally a linker (e.g., peptide linker) joining the variable heavy and variable light chain regions, wherein the variable heavy region comprises at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and wherein the variable light region comprises at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof. In one embodiment, the expression vector directs expression of the single chain antibody in the host cell.

The present disclosure also provides a host cell, or a population of host cells, wherein the host cell or individual host cells from the population of host cells harbors a first vector (e.g., a first expression vector) operably linked to a nucleic acid encoding a single chain fully human antibody comprising a polypeptide chain having a variable region from a fully human heavy chain and a variable region from a fully human light chain, and optionally a linker (e.g., peptide linker) joining the variable heavy and variable light chain regions, wherein the variable heavy region comprises at least 95% sequence identity, or at least 96% sequence identity, or at least 97% sequence identity, or at least 98% sequence identity, or at least 99% sequence identity to the amino acid sequence of SEQ ID NO:3, SEQ ID NO:5, SEQ ID NO:6, SEQ ID NO:7, SEQ ID NO:9, SEQ ID NO:10, SEQ ID NO:11 or SEQ ID NO:13, or combinations thereof, and wherein the variable light region comprises at least 95% sequence identity, or at least 96% sequence identity, or at least 97% sequence identity, or at least 98% sequence identity, or at least 99% sequence identity to the amino acid sequence of SEQ ID NO:4 or SEQ ID NO:12, or combinations thereof. In one embodiment, the expression vector directs expression of the single chain antibody in the host cell.

The present disclosure provides methods for inhibiting growth or proliferation of target cells, or methods for killing target cells (e.g., cytotoxicity), the method comprising: contacting a population of effector cells with a population of target cells (e.g., target cells expressing CD38) in the presence of any of the anti-CD38 variant antibodies (or variant antibody fragments thereof) described herein under conditions that are suitable for killing the target cells. In one embodiment, the population of effector cells comprises natural killer (NK) cells or peripheral blood mononuclear cells (PBMCs). The PBMCs can include lymphocytes, including T cells, B cells and/or NK cells. In one embodiment, the population of target cells comprise cells that express CD38, including Raji, Ramos, Daudi, MOLT-4, Karpas-707, REH, U-266/70, U-698, RPMI-8226, A549, B lymphocytes, CD4+ cells, CD8+ cells, or cells from a subject having a disease associated with CD38-expression. In one embodiment, the population of target cells are any type of transgenic cells that are engineered to express CD38. In one embodiment, the population of target cells comprise a cell line that is engineered to express CD38 such as CHO, HeLa, HEK293 or Panoply™ (from Creative Biogene, Shirley, N.Y.). In one embodiment, the ratio of effector to target cells can be about 1:1, or about 2:1, or about 3:1, or about 4:1, or about 5:1, or about 5-10:1, or about 10-20:1, or about 20-30:1.

The present disclosure provides methods for promoting phagocytosis of target cells, the method comprising: contacting a population of macrophage cells with a population of target cells (e.g., target cells expressing CD38) in the presence of any of the anti-CD38 variant antibodies (or variant antibody fragments thereof) described herein under conditions that are suitable for promoting phagocytosis of target cells. In one embodiment, the population of macrophage cells can be obtained by culturing monocytes with macrophage colony-stimulating factor (M-CSF) to promote proliferation and differentiation of the monocytes into macrophage cells. In one embodiment, the population of macrophage cells express CD14. In one embodiment, the population of target cells comprise cells that express CD38, including Raji, Ramos, Daudi, MOLT-4, Karpas-707, REH, U-266/70, U-698, RPMI-8226, A549, B lymphocytes, CD4+ cells, CD8+ cells, or cells from a subject having a disease associated with CD38-expression. In one embodiment, the population of target cells are any type of transgenic cells that are engineered to express CD38. In one embodiment, the population of target cells comprise a cell line that is engineered to express CD38 such as CHO, HeLa, HEK293 or PANOPLY. In one embodiment, the ratio of macrophage cells to target cells can be about 1:1, or about 2:1, or about 3:1, or about 4:1, or about 5:1, or about 5-10:1, or about 10-20:1, or about 20-30:1.

The present disclosure provides methods for treating a subject having a disease associated with CD38 expression (e.g., over-expression), the method comprising: administering to the subject an effective amount of a therapeutic composition comprising an anti-CD38 variant antibody or a variant antigen binding fragment thereof, which is selected from a group consisting of any of the fully human anti-CD38 antibodies described herein, any of the Fab fully human anti-CD38 antibodies described herein, and any of the single chain human anti-CD38 antibodies described herein. In one embodiment, the disease associated with CD38 expression is a hematological cancer, including leukemia, lymphoma, myeloma or B cell lymphoma. In one embodiment, the hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

### LIST OF SEQUENCES

Table 1 provides a listing of exemplary sequences disclosed herein. In one embodiment, an anti-CD38 antigen-binding protein comprises a heavy chain variable region wherein an SGR motif replaces an NGR motif; for example, in any one of SEQ ID NO:3, 5, 6, 7, 9, 10, 11 or 13, the NGR motif at positions 54-56 (bold and underlined in Table 1) may be replaced with an SGR motif.

TABLE 1

| Heavy chain variable region: | Light chain variable region: |
|---|---|
| A2 parental heavy chain | A2 parental light chain |
| SEQ ID NO: 1 | SEQ ID NO: 2 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREDWGGEFTDWGRGTLVTV | YCAAWDDSLSGYVFGSGTKVTVL |
| SS | |
| 3H10m1 SEQ ID NO: 3 | 3H10m1 SEQ ID NO: 4 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GFHFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREGWSGEFTDWGQGTLVTV | YCAAWDDSLSGYVFGSGTKVTVL |
| SS | |
| 3G8m1 SEQ ID NO: 5 | 3G8m1 SEQ ID NO: 4 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GFHFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREAWGGEFTNWGQGTLVTV | YCAAWDDSLSGYVFGSGTKVTVL |
| SS | |
| 3E3m1 SEQ ID NO: 6 | 3D3m1 SEQ ID NO: 4 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GFHFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREAWGGEFTDWGQGTLVTV | YCAAWDDSLSGYVFGSGTKVTVL |
| SS | |
| 3G3 SEQ ID NO: 7 | 3G3 SEQ ID NO: 2 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREAWSGEFTDWGQGTLVTV | YCAAWDDSLSGYVFGSGTKVTVL |
| SS | |
| 3E11 SEQ ID NO: 9 | 3E11 SEQ ID NO: 2 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREGWGGEFTDWGQGTLVTV | YCAAWDDSLSGYVFGSGTKVTVL |
| SS | |
| 3H10 SEQ ID NO: 10 | 3H10 SEQ ID NO: 2 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREGWSGEFTDWGQGTLVTV | YCAAWDDSLSGYVFGSGTKVTVL |
| SS | |
| 3H10N SEQ ID NO: 11 | 3H10N SEQ ID NO: 12 |
| QVQLVESGGGLVKPGGSLRLSCAASGFTF | QSVLTQPPSASGTSGQRVTISCSGSSSNIG |
| SDDYMSWIRQAPGKGLEWVASVSNGRPTT | FHFVYWYQHLPGTAPKLLIYKNNQRPSGVP |
| YYADSVRGRFTISRDNAKNSLYLQMNSLR | DRFSGSKSGNSASLAISGLRSEDEADYYCA |
| AEDTAVYYCAREDWGGEFTDWGQGTLVTV | AWDDSLSGYVFGSGTKVTVL |
| SS | |
| 3H10NS SEQ ID NO: 13 | 3H10NS SEQ ID NO: 12 |
| QVQLVESGGGLVKPGGSLRLSCAASGFTF | QSVLTQPPSASGTSGQRVTISCSGSSSNIG |
| SDDYMSWIRQAPGKGLEWVASVSSGRPTT | FHFVYWYQHLPGTAPKLLIYKNNQRPSGVP |
| YYADSVRGRFTISRDNAKNSLYLQMNSLR | DRFSGSKSGNSASLAISGLRSEDEADYYCA |
| AEDTAVYYCAREDWGGEFTDWGQGTLVTV | AWDDSLSGYVFGSGTKVTVL |
| SS | |
| 3E10 SEQ ID NO: 1 | 3E10 SEQ ID NO: 4 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GFHFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREDWGGEFTDWGRGTLVTV | YCAAWDDSLSGYVFGSGTKVTVL |
| SS | |
| 3H10m1g SEQ ID NO: 3 | 3H10m1g SEQ ID NO: 12 |
| QVQLVESGGGLVKPGGSLRLSCAASGFTF | QSVLTQPPSASGTSGQRVTISCSGSSSNIG |
| SDDYMSWIRQAPGKGLEWVASVSNGRPTT | FHFVYWYQHLPGTAPKLLIYKNNQRPSGVP |
| YYADSVRGRFTISRDNAKNSLYLQMNSLR | DRFSGSKSGNSASLAISGLRSEDEADYYCA |
| AEDTAVYYCAREGWSGEFTDWGQGTLVTV | AWDDSLSGYVFGSGTKVTVL |
| SS | |

TABLE 2
CDRs 1, 2 and 3:
A2 parent:
A2 (VH-CDR1) SEQ ID NO: 23 DDYMS
A2 (VH-CDR2) SEQ ID NO: 24 SVSNGRPTTYYADSVRG
A2 (VH-CDR3) SEQ ID NO: 25 EDWGGEFTD
A2 (VL-CDR1) SEQ ID NO: 26 SGSSSNIGINFVY
A2 (VL-CDR2) SEQ ID NO: 27 KNNQRPS
A2 (VL-CDR3) SEQ ID NO: 28 AAWDDSLSGYV
3H10m1:
3H10m1 (VH-CDR1) SEQ ID NO: 29 DDYMS
3H10m1 (VH-CDR2) SEQ ID NO: 30 SVSNGRPTTYYADSVRG
3H10m1 (VH-CDR3) SEQ ID NO: 31 EGWSGEFTD
3H10m1 (VL-CDR1) SEQ ID NO: 32 SGSSSNIGFHFVY
3H10m1 (VL-CDR2) SEQ ID NO: 33 KNNQRPS
3H10m1 (VL-CDR3) SEQ ID NO: 34 AAWDDSLSGYV
3G8m1:
3G8m1 (VH-CDR1) SEQ ID NO: 35 DDYMS
3G8m1 (VH-CDR2) SEQ ID NO: 36 SVSNGRPTTYYADSVRG
3G8m1 (VH-CDR3) SEQ ID NO: 37 EAWGGEFTN
3G8m1 (VL-CDR1) SEQ ID NO: 38 SGSSSNIGFHFVY
3G8m1 (VL-CDR2) SEQ ID NO: 39 KNNQRPS
3G8m1 (VL-CDR3) SEQ ID NO: 40 AAWDDSLSGYV
3E3m1:
3E3m1 (VH-CDR1) SEQ ID NO: 41 DDYMS
3E3m1 (VH-CDR2) SEQ ID NO: 42 SVSNGRPTTYYADSVRG
3E3m1 (VH-CDR3) SEQ ID NO: 43 EAWGGEFTD
3E3m1 (VL-CDR1) SEQ ID NO: 44 SGSSSNIGFHFVY
3E3m1 (VL-CDR2) SEQ ID NO: 45 KNNQRPS
3E3m1 (VL-CDR3) SEQ ID NO: 46 AAWDDSLSGYV
3G3:
3G3 (VH-CDR1) SEQ ID NO: 47 DDYMS
3G3 (VH-CDR2) SEQ ID NO: 48 SVSNGRPTTYYADSVRG
3G3 (VH-CDR3) SEQ ID NO: 49 EAWSGEFTD
3G3 (VL-CDR1) SEQ ID NO: 50 SGSSSNIGINFVY
3G3 (VL-CDR2) SEQ ID NO: 51 KNNQRPS
3G3 (VL-CDR3) SEQ ID NO: 52 AAWDDSLSGYV
3E11:
3E11 (VH-CDR1) SEQ ID NO: 53 DDYMS
3E11 (VH-CDR2) SEQ ID NO: 54 SVSNGRPTTYYADSVRG
3E11 (VH-CDR3) SEQ ID NO: 55 EGWGGEFTD
3E11 (VL-CDR1) SEQ ID NO: 56 SGSSSNIGINFVY
3E11 (VL-CDR2) SEQ ID NO: 57 KNNQRPS
3E11 (VL-CDR3) SEQ ID NO: 58 AAWDDSLSGYV
3H10:
3H10 (VH-CDR1) SEQ ID NO: 59 DDYMS
3H10 (VH-CDR2) SEQ ID NO: 60 SVSNGRPTTYYADSVRG
3H10 (VH-CDR3) SEQ ID NO: 61 EGWSGEFTD
3H10 (VL-CDR1) SEQ ID NO: 62 SGSSSNIGINFVY
3H10 (VL-CDR2) SEQ ID NO: 63 KNNQRPS
3H10 (VL-CDR3) SEQ ID NO: 64 AAWDDSLSGYV
3H10N:
3H10N (VH-CDR1) SEQ ID NO: 65 DDYMS
3H10N (VH-CDR2) SEQ ID NO: 66 SVSNGRPTTYYADSVRG
3H10N (VH-CDR3) SEQ ID NO: 67 EDWGGEFTD
3H10N (VL-CDR1) SEQ ID NO: 68 SGSSSNIGFHFVY
3H10N (VL-CDR2) SEQ ID NO: 69 KNNQRPS
3H10N (VL-CDR3) SEQ ID NO: 70 AAWDDSLSGYV
3H10NS:
3H10NS (VH-CDR1) SEQ ID NO: 71 DDYMS
3H10NS (VH-CDR2) SEQ ID NO: 72 SVSSGRPTTYYADSVRG
3H10NS (VH-CDR3) SEQ ID NO: 73 EDWGGEFTD
3H10NS (VL-CDR1) SEQ ID NO: 74 SGSSSNIGFHFVY
3H10NS (VL-CDR2) SEQ ID NO: 75 KNNQRPS
3H10NS (VL-CDR3) SEQ ID NO: 76 AAWDDSLSGYV
3E10:
3E10 (VH-CDR1) SEQ ID NO: 77 DDYMS
3E10 (VH-CDR2) SEQ ID NO: 78 SVSNGRPTTYYADSVRG
3E10 (VH-CDR3) SEQ ID NO: 79 EDWGGEFTD
3E10 (VL-CDR1) SEQ ID NO: 80 SGSSSNIGFHFVY
3E10 (VL-CDR2) SEQ ID NO: 81 KNNQRPS
3E10 (VL-CDR3) SEQ ID NO: 82 AAWDDSLSGYV
3H10m1g:
3H10m1g (VH-CDR1) SEQ ID NO: 83 DDYMS
3H10m1g (VH-CDR2) SEQ ID NO: 84 SVSNGRPTTYYADSVRG
3H10m1g (VH-CDR3) SEQ ID NO: 85 EGWSGEFTD
3H10m1g (VL-CDR1) SEQ ID NO: 86 SGSSSNIGFHFVY
3H10m1g (VL-CDR2) SEQ ID NO: 87 KNNQRPS
3H10m1g (VL-CDR3) SEQ ID NO: 88 AAWDDSLSGYV

### ADDITIONAL SEQUENCES

Anti-CD38 IgG1: heavy chain constant region:
SEQ ID NO: 14
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Anti-CD38 IgG1-SPPC: heavy chain constant region:
SEQ ID NO: 15
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTSPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Anti-CD38 IgG4: heavy chain constant region:
SEQ ID NO: 16
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Anti-CD38: lambda light chain constant region:
SEQ ID NO: 17
GQPKAAPSVTLFPPSSEELQANKATLVCLISDFYPGAVTVAWKADSSPVK
AGVETTTPSKQSNNKYAASSYLSLIPEQWKSHRSYSCQVTHEGSTVEKTV
APTECS
Anti-CD38: kappa light chain constant region:
SEQ ID NO: 18
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
CD38 antigen-human (UniProt P28907):
SEQ ID NO: 19
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI
CD38 antigen-cynomolgus monkey (UniProt Q5VAN0:
SEQ ID NO: 20
MANCEFSPVSGDKPCCRLSRRAQVCLGVCLLVLLILVVVVAVVLPRWRQQ
WSGSGTTSRFPETVLARCVKYTEVHPEMRHVDCQSVWDAFKGAFISKYPC
NITEEDYQPLVKLGTQTVPCNKTLLWSRIKDLAHQFTQVQRDMFTLEDML
LGYLADDLTWCGEFNTFEINYQSCPDWRKDCSNNPVSVFWKTVSRRFAET
ACGVVHVMLNGSRSKIFDKNSTFGSVEVHNLQPEKVQALEAWVIHGGRED
SRDLCQDPTIKELESIISKRNIRFFCKNIYRPDKFLQCVKNPEDSSCLSG
CD38 antigen-mouse (UniProt P56528:
SEQ ID NO: 21
MANYEFSQVSGDRPGCRLSRKAQIGLGVGLLVLIALVVGIVVILLRPRSL
LVWTGEPTTKHFSDIFLGRCLIYTQILRPEMRDQNCQEILSTFKGAFVSK
NPCNITREDYAPLVKLVTQTIPCNKTLFWSKSKHLAHQYTWIQGKMFTLE
DTLLGYIADDLRWCGDPSTSDMNYVSCPHWSENCPNNPITVFWKVISQKF
AEDACGVVQVMLNGSLREPFYKNSTFGSVEVFSLDPNKVHKLQAWVMHDI
EGASSNACSSSSLNELKMIVQKRNMIFACVDNYRPARFLQCVKNPEHPSC
RLNT
CD38 antigen-rat (UniProt Q64244:
SEQ ID NO: 22
MANYEFSQVSEDRPGCRLTRKAQIGLGVGLLLLVALVVVVVIVLWPRSPL
VWKGKPTTKHFADIILGRCLIYTQILRPEMRDQDCKKILSTFKRGFISKN
PCNITNEDYAPLVKLVTQTIPCNKTLFWSKSKHLAHQYTWIQGKMFTLED
TLLGYIADDLRWCGDPSTSDMNYDSCPHWSENCPNNPVAVFWNVISQKFA
EDACGVVQVMLNGSLSEPFYRNSTFGSVEVFNLDPNKVHKLQAWVMHDIK
GTSSNACSSPSINELKSIVNKRNMIFACQDNYRPVRFLQCVKNPEHPSCR
LNV

### EXAMPLES

The following examples are meant to be illustrative and can be used to further understand embodiments of the present disclosure, and should not be construed as limiting the scope of the present teachings in any way.

### Example 1: Comparative Antigen Binding Analysis of Parent Antibody A2 and Variant Antibodies

Binding kinetics of A2 parent antibody, variant antibodies and a commercially-available anti-CD38 antibody Daratumumab (Darzalex) was measured using surface plasmon resonance (SPR). Kinetic interaction between a his-tagged CD38 protein and the various anti-CD38 variant antibodies was measured at approximately 25° C. using a Biacore T200 surface plasmon resonance (GE Healthcare).

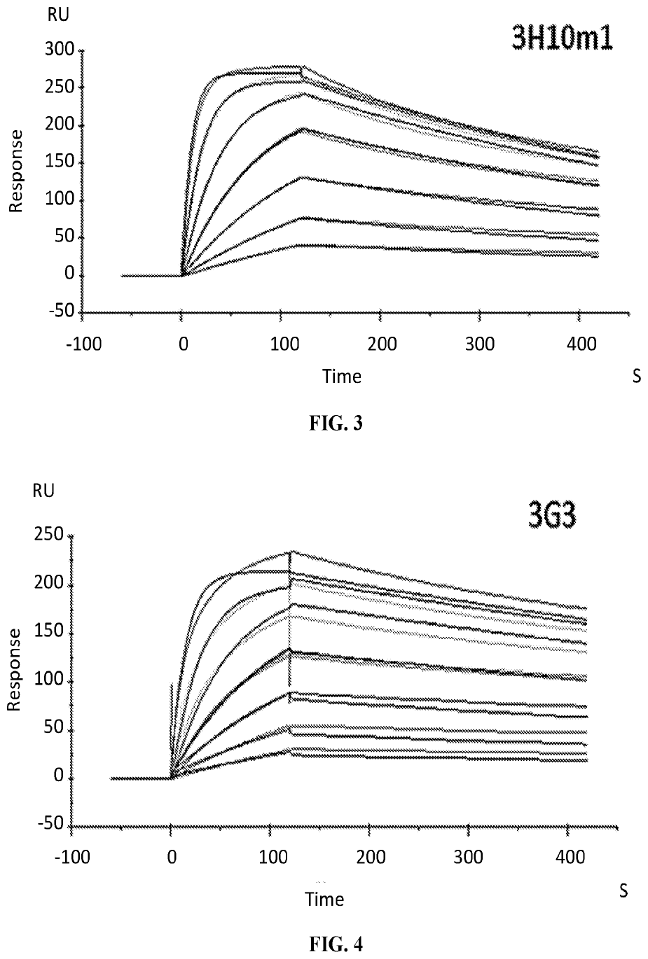

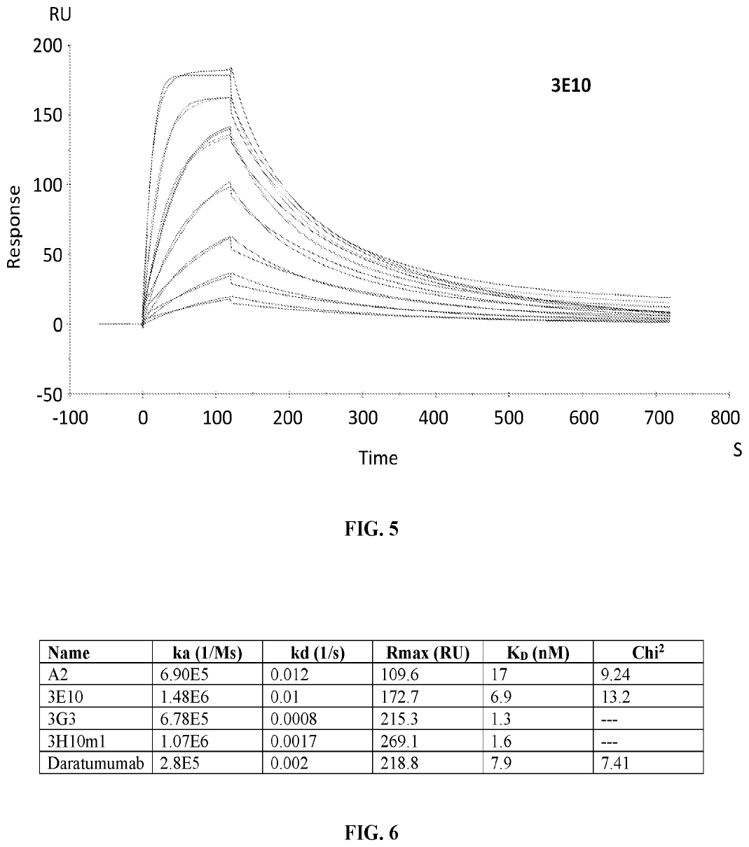

Anti-human Fc antibody from Human Antibody Capture Kit (catalog #BR-1008-39, from GE Healthcare) was immobilized on a Series S Sensor Chip CM5 (catalog #BR-1005-30, GE Healthcare) to approximately 5000 resonance units (RU) using standard N-Hydroxysuccinimide/1-ethyl-3-(-3-dimethylaminopropyl) carbodiimide hydrochloride (NHS/EDC) coupling methodology. Anti-CD38 antibodies (approximately 2 μg/mL) were captured for 60 seconds at a flow rate of approximately 10 μL/minute. Recombinant human his-tagged CD38 protein (from Sino Biological, catalog #10818-H08H) was serially diluted in running buffer of 0.01 M HEPES pH 7.4, 0.15 M NaCl, 3 mM EDTA, 0.05% v/v Surfactant P20 (HBS EP+). All measurements were conducted in HBS-EP+ buffer with a flow rate of 30 μL/minute. Surfaces were regenerated with 3M MgCl₂ for 60 seconds. A 1:1 (Langmuir) binding model was used to fit the data. The SPR sensorgrams are shown in FIG. 1 (Daratumumab, from Janssen Biotech), FIG. 2 (parent antibody A2), FIG. 3 (variant antibody 3H10m1), FIG. 4 (variant antibody 3G3), and FIG. 5 (variant antibody 3E10). The results of the binding assays, including binding kinetics, are summarized in the table shown in FIG. 6. The binding assays show that the variant antibodies 3E10, 3G3 and 3H10m1 exhibit improved affinity to CD38 protein compared to the parent antibody A2, and these variant antibodies exhibit higher affinity for CD38 protein compared to Daratumumab, as indicated by their measured K_{D} values.

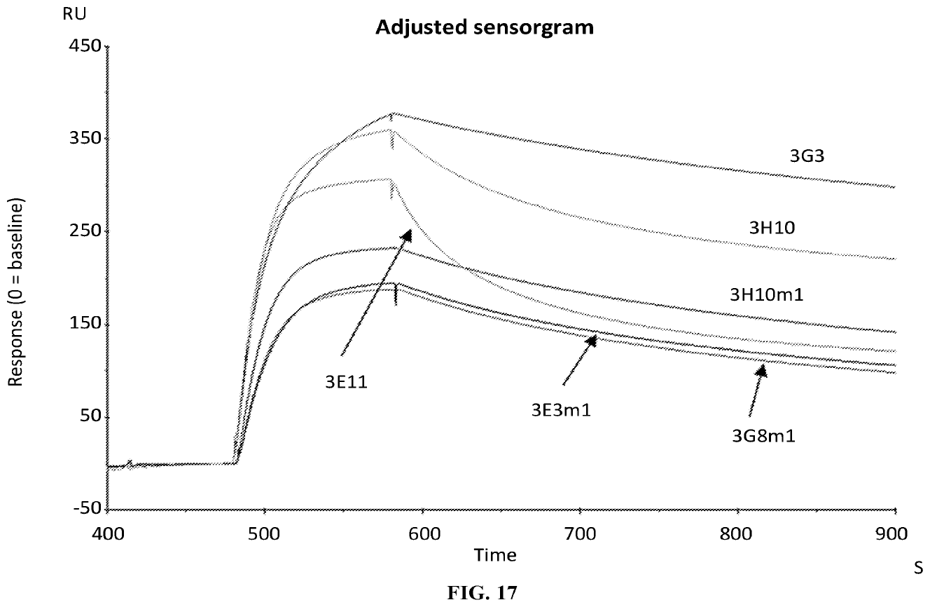

Surface plasmon resonance was also used to rank affinities of six variant antibodies (variant antibody 3E10 was not included in this analysis). The SPR sensorgram at FIG. 17 shows the ranked order, from highest to lowest affinity, 3G3>3H10>3H10m1>3E11>3E3m1>3G8m1.

### Example 2: Comparative Cell Binding Analysis of Parent Antibody A2 and Variant Antibodies

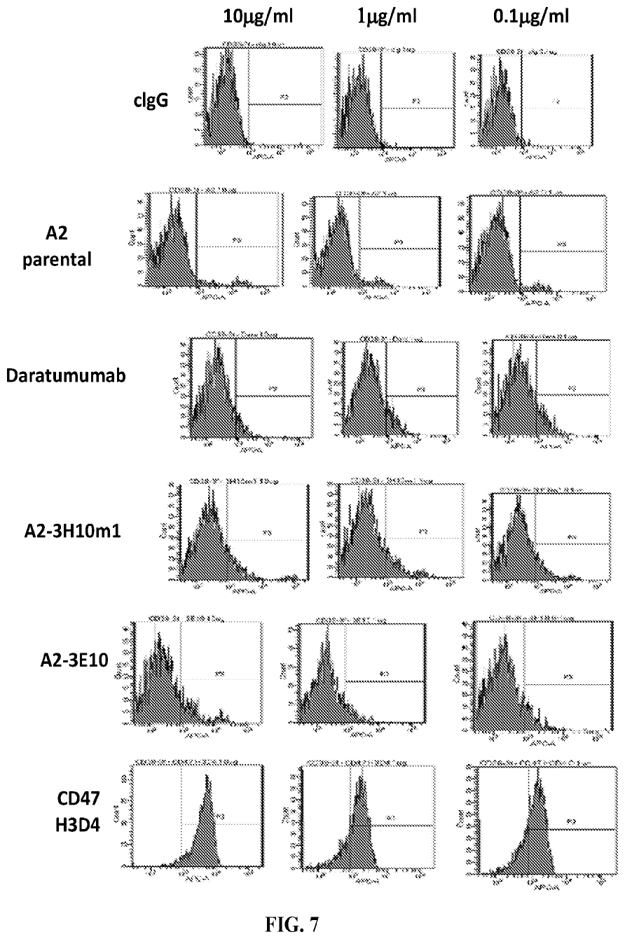

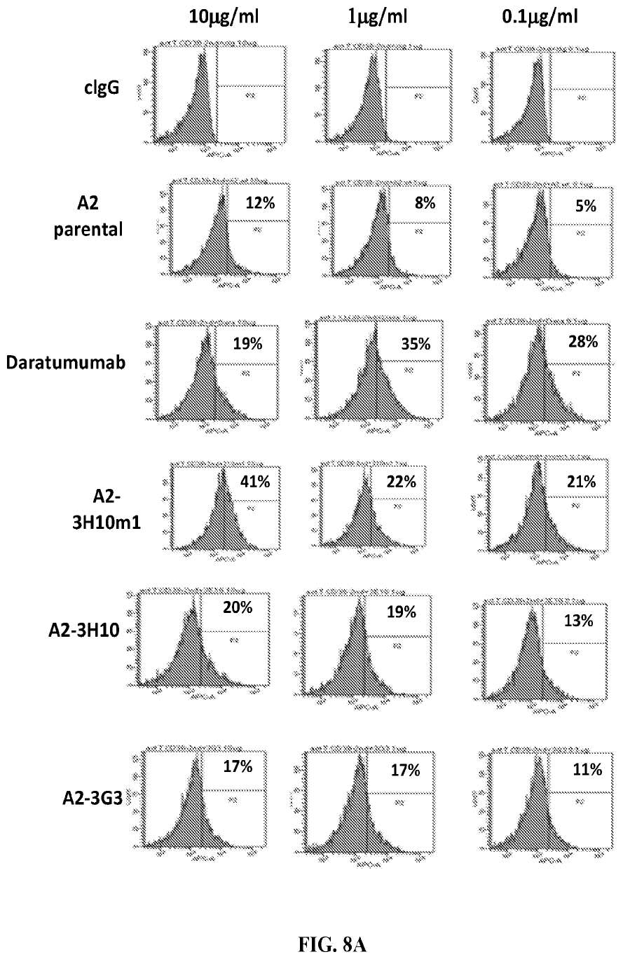

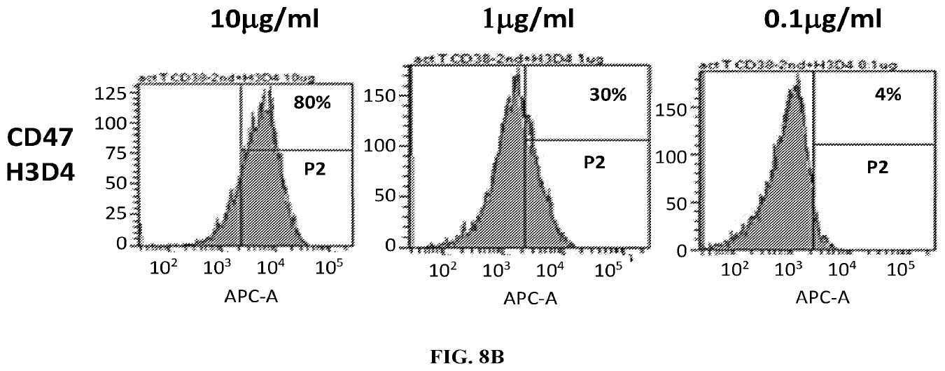

The capability of the parent antibody A2, variant antibodies and Daratumumab to bind non-activated or activated T cells, was measured using flow cytometry. Serial dilutions of the antibodies, at 10 μg/mL, 1 μg/mL or 0.1 μg/mL, were added to 1×10⁵ human T cells (non-activated or activated) in a final volume of 100 μL of PBS+2% FCS in the well of a 96-well plate. After 15 minutes at 4° C., the wells were washed with a solution of PBS+2% FCS. The cells were resuspended in a 100 μL solution of PBS+2% FCS containing the APC-labeled goat anti-human IgG at a final concentration of 1:1,000. After 15 minutes at 4° C., the cells were washed with a solution of PBS+2% FCS and then resuspended in 150 μL of PBS+2% FCS for analysis by flow cytometry. Controls for these cell binding assays included (1) T cells incubated with an irrelevant isotype control antibody followed by incubation with the APC-labeled goat anti-human IgG, and (2) T cells stained with an antiCD47 antibody. The results shown in FIGS. 7, 8A and 8B demonstrate that variant antibodies 3H10m1, 3G3 and 3E10 exhibit improved cell binding capabilities compared to parent antibody A2 at all concentrations tested, and these variant antibodies have cell binding capabilities that are comparable to Daratumumab.

### Example 3: Comparative Cell Binding Analysis of Parent Antibody A2 and Variant Antibodies

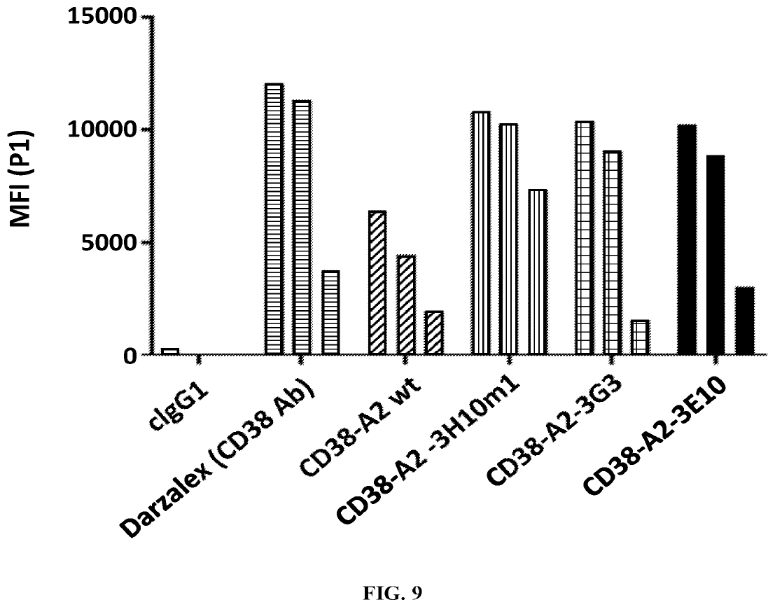

The capability of the parent antibody A2, variant antibodies, and Daratumumab to bind RPMI 8226 cells was measured using flow cytometry. The antibody serial dilutions were prepared as described in Example 2 above. The diluted antibodies were added to 1×10⁵ RPMI 8226 cells in a manner similar to the procedure described in Example 2 above. The results in FIG. 9 demonstrate that variant antibodies 3H10m1, 3G3 and 3E10 exhibit improved cell binding capabilities compared to parent antibody A2 at all concentrations tested, and these variant antibodies have cell binding capabilities that are comparable to Daratumumab.

### Example 4: Comparative Cell Binding Analysis of Parent Antibody A2 and Variant Antibodies

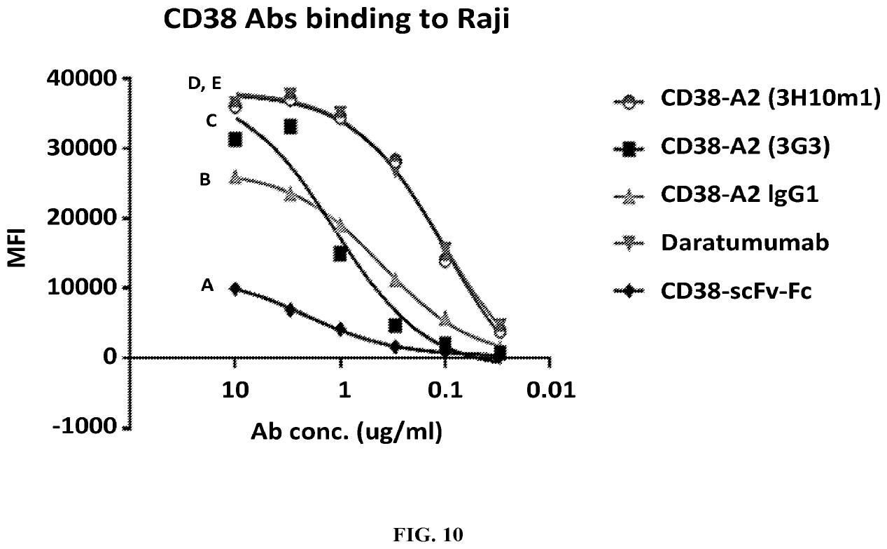

The capability of the parent antibody A2, variant antibodies, and Daratumumab to bind B lymphoma cells, was measuring using flow cytometry. Serial dilutions of the antibodies, at 10 μg/mL, 1 μg/mL, 0.1 μg/mL or 0.01 μg/mL, were prepared as described in Example 2 above. The diluted antibodies were added to 1×10⁵Raji (FIG. 9) or Ramos (FIG. 10) cells in a manner similar to the procedure described in Example 2 above.

The results in FIG. 10 and Table 3 below show that the EC50 of variant antibody 3H10m1 is lower than that of the parent antibody A2 when binding to Raji cells and is comparable to the EC50 of Daratumumab.

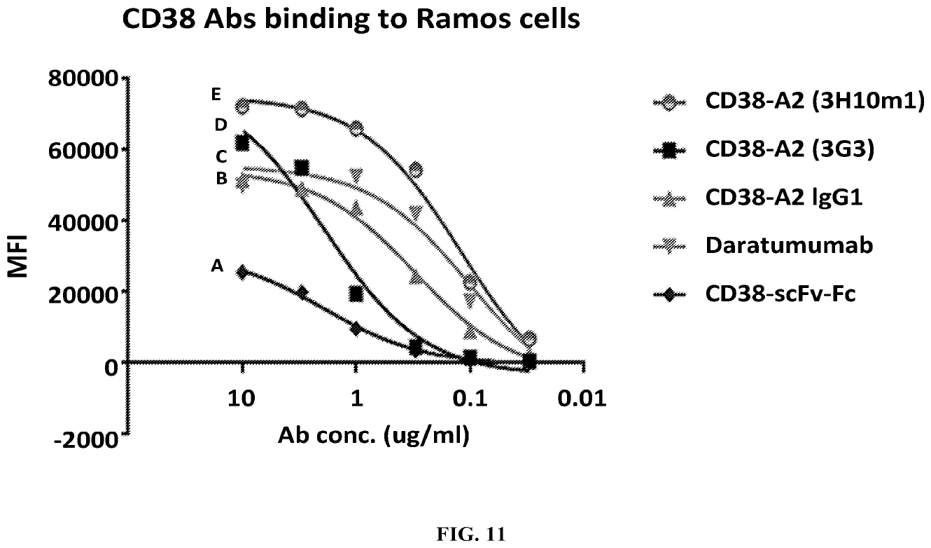

The results in FIG. 11 and Table 4 below show that the EC50 of variant antibody 3H10m1 is lower than that of the parent antibody A2 when binding to Ramos cells and is comparable to the EC50 of Daratumumab.

TABLE 3

| | A2-IgG1 | scFv-Fc | 3H10m1 | 3G3 | Daratumumab |
|---|---|---|---|---|---|
| EC50 | 0.4039 | 2.152 | 0.09775 | 1.128 | 0.1061 |

TABLE 4

| | A2-IgG1 | scFv-Fc | 3H10m1 | 3G3 | Daratumumab |
|---|---|---|---|---|---|
| EC50 | 0.302 | 1.883 | 0.1196 | 1.9 | 0.1023 |

### Example 5: Comparative Cell Binding Analysis of Parent Antibody A2 and Variant Antibodies

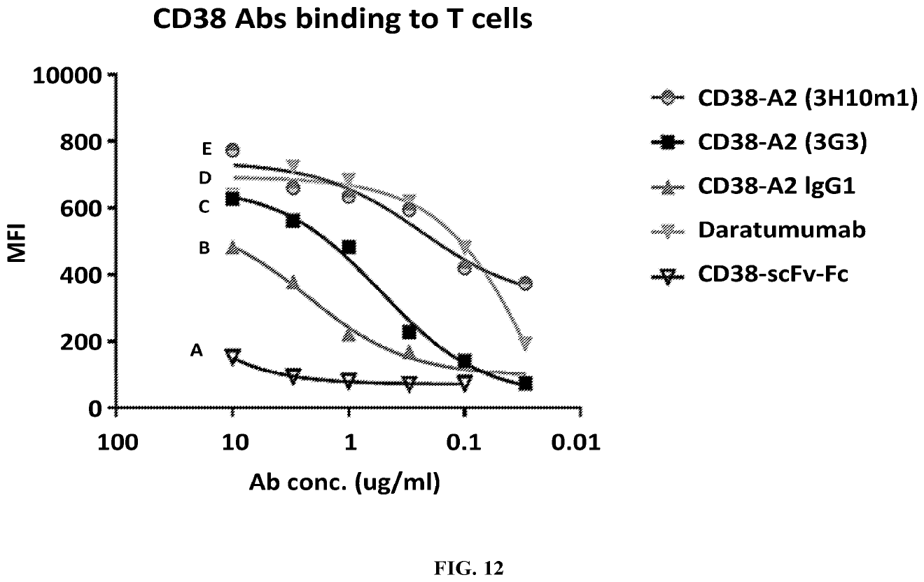

The capability of the parent antibody A2, variant antibodies, and Daratumumab to bind primary T cells was measuring using flow cytometry. Serial dilutions of the antibodies, at 10 μg/mL, 1 μg/mL, 0.1 μg/mL or 0.01 μg/mL, were prepared as described in Example 2 above. The diluted antibodies were added to 1×10⁵ human primary T cells (FIG. 12) in a manner similar to the procedure described in Example 2 above.

The results in FIG. 12 and Table 5 below show that the EC50 of variant antibodies 3H10m1 and 3G3 is lower than that of the parent antibody A2 when binding to primary T cells but not as low as the EC50 of Daratumumab.

TABLE 5

| | A2-IgG1 | 3H10m1 | 3G3 | Daratumumab |
|---|---|---|---|---|
| EC50 | 2.239 | 0.2361 | 0.5307 | 0.01892 |

### Example 6: Cross-Reactivity Analysis of Parent Antibody A2 and Daratumumab

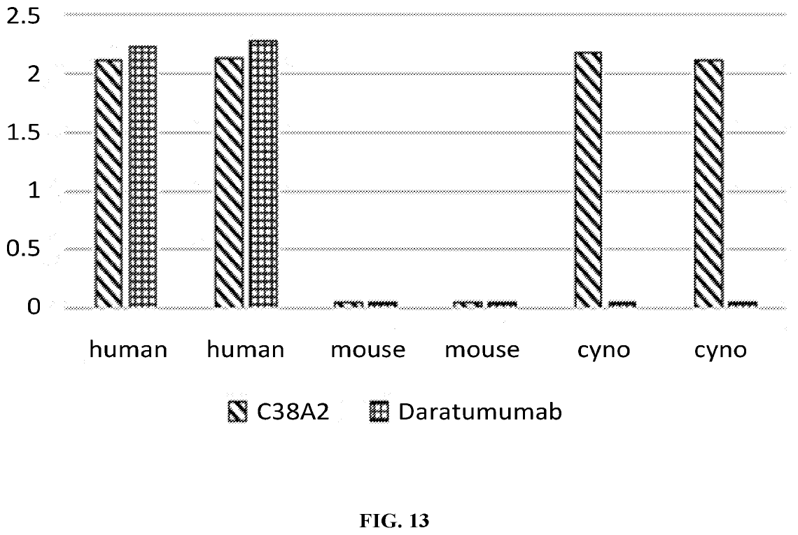

The capability of parent antibody A2 and Daratumumab to bind to murine and cynomolgus CD38 protein was analyzed. A 96-well Ni-NTA plate was used to capture 50 μL recombinant human (Sino Biological, catalog #10818-H08H), mouse (Sino Biological, catalog #50191-M08H), or cynomolgus (Sino Biological, catalog #90050-C08H) CD38/His-tag (1 μg/mL in PBS). After incubating for 30 minutes at room temperature, the wells were washed 3 times with PBS-0.05% Tween 20 (PBST). 50 μL of the antibody (about 1 μg/mL) diluted in Casein were added and incubated for 30 minutes with shaking at room temperature. The plate was washed 3 times with PBST followed by a 30 minute incubation with 50 μL horseradish peroxidase (HRP)-conjugated goat anti-human Fc (1:1000 in casein) for 30 minutes. After washing, 25 μL 3,3′,5,5′-Tetramethylbenzidine (TMB) substrate was added and developed for 30 minutes at room temperature. 25 μL 2M H₂SO₄ was used to stop the reaction and the OD was read at 450 nm. The graph shown in FIG. 13 shows that the parent antibody A2 and Daratumumab bind human CD38 protein but not murine CD38 protein. Notably, the parent antibody A2 binds cynomolgus CD38 protein whereas Daratumumab does not. This result indicates that the parent antibody A2 can enable toxicology evaluation in non-human primates for CD38-based immunotherapy studies.

### Example 7: Cross-Reactivity Analysis of Parent Antibody A2 and Variant Antibodies

The capability of parent antibody A2, variant antibodies 3H10m1 and 3G3, and Daratumumab, to bind to cynomolgus monkey T cells was analyzed.

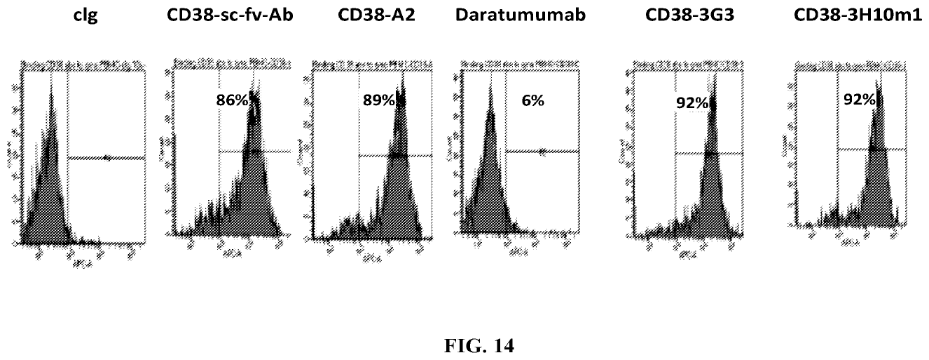

Peripheral blood lymphocytes from cynomolgus monkey blood were stained with an anti-CD3 FITC antibody plus the indicated anti-CD38 antibody. All reagents except Daratumumab were reactive with cynomolgus T cells. The results in FIG. 14 show that variant antibodies 3H10m1 and 3G3 bind cynomolgus T cells but Daratumumab does not.

### Example 8: ADCP Analysis of Parent Antibody A2 and Variant Antibodies

The capability of the parent antibody A2, variant antibodies 3H10m1 and 3G3, and Daratumumab to exhibit antibody-dependent cellular phagocytic (ADCP) activity were analyzed.

Macrophages were prepared by culturing purified human monocytes with macrophage colony-stimulating factor (M-CSF) at 30 ng/mL for 6-7 days. Monocytes were obtained from peripheral blood mononuclear cells (PBMCs) by reactivity to biotin anti-human CD14, followed the addition of magnetic anti-biotin beads and positive selection of the labeled population by passage over a column attached to a magnet. This typically resulted in a population of >90% pure CD14 positive monocytes.

On the day prior to the phagocytosis assay, cells from the Burkitt B lymphoma cell line Ramos were labeled with the fluorescent dye carboxyfluorescein succinimidyl ester (CFSE). Ramos cells were harvested and centrifuged at 500g for 5 minutes and the pelleted cells were resuspended in 900 μL of PBS. 100 μL of a solution of CFSE at 5 μM was then added to the cells (final CFSE concentration: 500 nM). After 8 minutes at 37° C., the cells were washed twice with RPMI+10% FCS and then re-cultured.

To measure phagocytic activity, 1×10⁵ macrophages were cultured with 5×10³ Ramos cells in the wells of 96-well plate in the presence or absence of an anti-CD38 antibody at 10 per mL. After three hours of incubation at 37° C. the cells were stained with phycoerythrin (PE)-conjugated anti-human CD11b to identify the macrophages. The cells were then analyzed by flow cytometry with phagocytic activity being detected as cells which were positive for both CD11b staining and CFSE labeling.

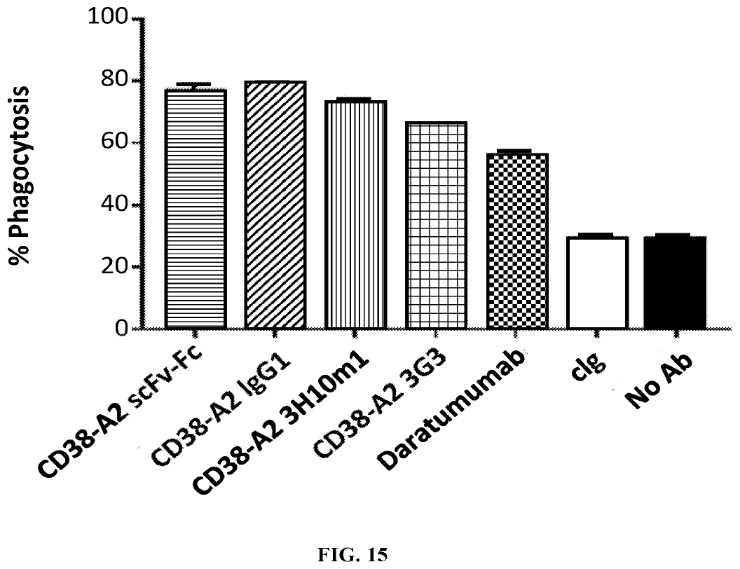

The bar graph shown in FIG. 15 demonstrates that the parent antibody A2, and variant antibodies 3H10m1 and 3G3, exhibited higher levels of ADCP activity compared to Daratumumab.

### Example 9: ADCC Analysis of Parent Antibody A2 and Variant Antibodies

The capability of the parent antibody A2, variant antibodies 3H10m1 and 3G3, and Daratumumab to exhibit antibody-dependent cellular cytotoxicity (ADCC) activity was analyzed.

NK cells served as the effector population in the ADCC assay. The NK cells were prepared from peripheral blood mononuclear cells (PBMC) using a human NK cell enrichment kit (StemCell Technologies). Once prepared, the NK cells were cultured in RPMI+10% FCS containing IL-2 (100 U/mL). After 1 day of culture at 37° C. and 5% CO₂, the NK cells were harvested, washed by centrifugation at 500g for 5 minutes, then resuspended in RPMI+10% FCS and counted using a hemocytometer

The anti-CD38 antibodies were added at 10 μg/mL or indicated dilutions thereof to 5×10³ Raji or 5×10³ T cells in 100 μL RPMI+10% FCS in the wells of a 96-well white plate. After incubating 20 minutes at 37° C. and 5% CO₂, the cells were washed with RPMI+2% FCS followed by the addition of 1.5×10⁵ NK cells to give an effector to target ratio of 10:1. The cells were incubated at 37° C. and 5% CO₂ for a minimum of 4 hours, followed by measurement of cytotoxicity using the CytoTox-Glo™ kit per manufacturer's instructions and determining the luminescence on a FlexStation. Controls consisted of cells treated as above except that no primary antibody was added, and cells treated as above except an irrelevant isotype-matched control antibody was used instead of an anti-CD38 antibody.

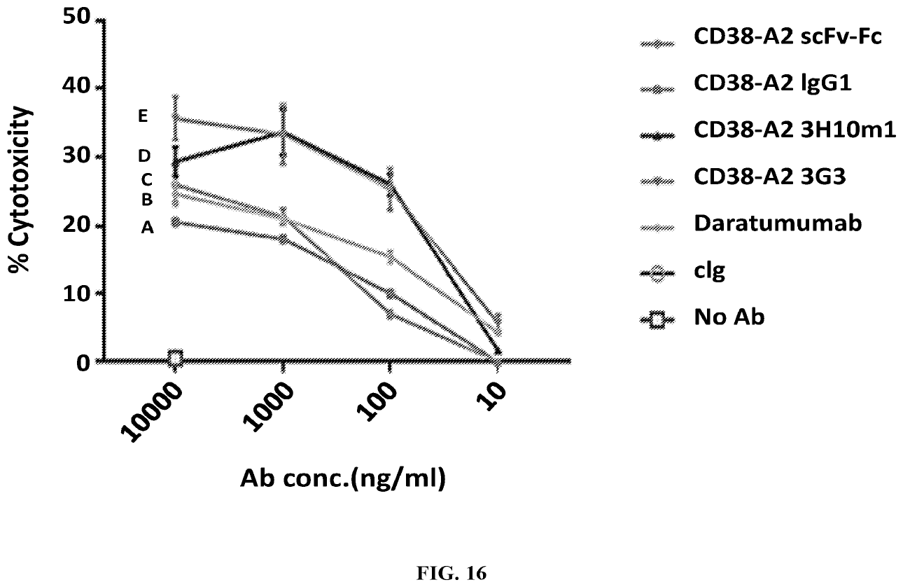

The graph at FIG. 16 shows that variant antibodies 3H10m1 (line D) and 3G3 (line E) exhibit a greater ability to promote cytotoxicity compared to the parent antibody A2 (line A) and Daratumumab (line B).

### Example 10: Thermal Stability of Parent Antibody A2 and Variant Antibodies

UNcle from Unchained Labs was used to measure thermal stability in terms of Tm using fluorescence. In a typical run, 9 μL of antibody with a concentration range of 0.1 mg/mL to 100 mg/mL was loaded. The thermal ramping was performed from 20° C. to 90° C. at a scan rate of 1° C./min, and fluorescence at full 250-720 nm spectral range was captured using a CCD digital camera. The UNcle software automatically displays the fluorescence curve calculated by BCM, and the midpoint of a thermal transition temperature (Tm, or thermal transition temperature). The results are summarized in Table 6 below.

TABLE 6

| IgG1 | Tm |
|---|---|
| A2 | 70.6 |
| 3H10m1 | 67.9 |
| 3G3 | 69.2 |

What is claimed:

1. An anti-CD38 antigen-binding protein or fully human anti-CD38 antibody, or an antigen-binding fragment thereof, comprising a heavy chain variable region and a light chain variable region,
wherein the heavy chain variable region comprises a heavy chain complementarity determining region 1 (CDR1) a heavy chain CDR2 and a heavy chain CDR3, and the light chain variable region comprises a light chain CDR1, a light chain CDR2, and a light chain CDR3; and
(a) the heavy chain CDR1 has the amino acid sequence of SEQ ID NO:29, the heavy chain CDR2 has the amino acid sequence of SEQ ID NO:30, the heavy chain CDR3 has the amino acid sequence of SEQ ID NO:31, the light chain CDR1 has the amino acid sequence of SEQ ID NO:32, the light chain CDR2 has the amino acid sequence of SEQ ID NO:33, and the light chain CDR3 has the amino acid sequence of SEQ ID NO:34; (b) the heavy chain CDR1 has the amino acid sequence of SEQ ID NO:35, the heavy chain CDR2 has the amino acid sequence of SEQ ID NO:36, the heavy chain CDR3 has the amino acid sequence of SEQ ID NO:37, the light chain CDR1 has the amino acid sequence of SEQ ID NO:38, the light chain CDR2 has the amino acid sequence of SEQ ID NO:39, and the light chain CDR3 has the amino acid sequence of SEQ ID NO:40; (c) the heavy chain CDR1 has the amino acid sequence of SEQ ID NO:41, the heavy chain CDR2 has the amino acid sequence of SEQ ID NO:42, the heavy chain CDR3 has the amino acid sequence of SEQ ID NO:43, the light chain CDR1 has the amino acid sequence of SEQ ID NO:44, the light chain CDR2 has the amino acid sequence of SEQ ID NO:45, and the light chain CDR3 has the amino acid sequence of SEQ ID NO:46; (d) the heavy chain CDR1 has the amino acid sequence of SEQ ID NO:47, the heavy chain CDR2 has the amino acid sequence of SEQ ID NO:48, the heavy chain CDR3 has the amino acid sequence of SEQ ID NO:49, the light chain CDR1 has the amino acid sequence of SEQ ID NO:50, the light chain CDR2 has the amino acid sequence of SEQ ID NO:51, and the light chain CDR3 has the amino acid sequence of SEQ ID NO:52; (e) the heavy chain CDR1 has the amino acid sequence of SEQ ID NO:53, the heavy chain CDR2 has the amino acid sequence of SEQ ID NO:54, the heavy chain CDR3 has the amino acid sequence of SEQ ID NO:55, the light chain CDR1 has the amino acid sequence of SEQ ID NO:56, the light chain CDR2 has the amino acid sequence of SEQ ID NO:57, and the light chain CDR3 has the amino acid sequence of SEQ ID NO:58; (f) the heavy chain CDR1 has the amino acid sequence of SEQ ID NO:59, the heavy chain CDR2 has the amino acid sequence of SEQ ID NO:60, the heavy chain CDR3 has the amino acid sequence of SEQ ID NO:61, the light chain CDR1 has the amino acid sequence of SEQ ID NO:62, the light chain CDR2 has the amino acid sequence of SEQ ID NO:63, and the light chain CDR3 has the amino acid sequence of SEQ ID NO:64; (g) the heavy chain CDR1 has the amino acid sequence of SEQ ID NO:65, the heavy chain CDR2 has the amino acid sequence of SEQ ID NO:66, the heavy chain CDR3 has the amino acid sequence of SEQ ID NO:67, the light chain CDR1 has the amino acid sequence of SEQ ID NO:68, the light chain CDR2 has the amino acid sequence of SEQ ID NO:69, and the light chain CDR3 has the amino acid sequence of SEQ ID NO:70; (h) the heavy chain CDR1 has the amino acid sequence of SEQ ID NO:71, the heavy chain CDR2 has the amino acid sequence of SEQ ID NO:72, the heavy chain CDR3 has the amino acid sequence of SEQ ID NO:73, the light chain CDR1 has the amino acid sequence of SEQ ID NO:74, the light chain CDR2 has the amino acid sequence of SEQ ID NO:75, and the light chain CDR3 has the amino acid sequence of SEQ ID NO:76; (i) the heavy chain CDR1 has the amino acid sequence of SEQ ID NO:77, the heavy chain CDR2 has the amino acid sequence of SEQ ID NO:78, the heavy chain CDR3 has the amino acid sequence of SEQ ID NO:79, the light chain CDR1 has the amino acid sequence of SEQ ID NO:80, the light chain CDR2 has the amino acid sequence of SEQ ID NO:81, and the light chain CDR3 has the amino acid sequence of SEQ ID NO:82; or (j) the heavy chain CDR1 has the amino acid sequence of SEQ ID NO:83, the heavy chain CDR2 has the amino acid sequence of SEQ ID NO:84, the heavy chain CDR3 has the amino acid sequence of SEQ ID NO:85, the light chain CDR1 has the amino acid sequence of SEQ ID NO:86, the light chain CDR2 has the amino acid sequence of SEQ ID NO:87, and the light chain CDR3 has the amino acid sequence of SEQ ID NO:88.

2. The antigen-binding protein, antibody or antigen-binding fragment thereof of claim 1, wherein the heavy chain variable region has at least 95% sequence identity to the amino acid sequence of SEQ ID NO:3, 5, 6, 7, 9, 10, 11 or 13, and the light chain variable region has at least 95% sequence identity to the amino acid sequence of SEQ ID NO:4 or 12.

3. An antigen-binding protein or fully human anti-CD38 antibody, or an antigen-binding fragment thereof, comprising a heavy chain variable region and a light chain variable region, the heavy chain variable region having at least 95% sequence identity to the amino acid sequence of SEQ ID NO:3, 5, 6, 7, 9, 10, 11 or 13, and the light chain variable region having at least 95% sequence identity to the amino acid sequence of SEQ ID NO:4 or 12.

4. An antigen-binding protein or fully human anti-CD38 antibody, or an antigen-binding fragment thereof, comprising a heavy chain variable region and a light chain variable region, wherein the heavy chain variable region and the light chain variable region comprise the amino acid sequences of SEQ ID NOS:3 and 4, respectively (e.g., herein called 3H10m1); SEQ ID NOS:5 and 4, respectively (e.g., herein called 3G8m1); SEQ ID NOS:6 and 4, respectively (e.g., herein called 3E3m1); SEQ ID NOS:7 and 2, respectively (e.g., herein called 3G3); SEQ ID NOS:9 and 2, respectively (e.g., herein called 3E11); SEQ ID NOS:10 and 2, respectively (e.g., herein called 3H10); SEQ ID NOS:11 and 12, respectively (e.g., herein called 3H10N); SEQ ID NOS:13 and 12, respectively (e.g., herein called 3H10NS); SEQ ID NOS:1 and 4, respectively (e.g., herein called 3E10); or SEQ ID NOS:3 and 12, respectively (e.g., herein called 3H10m1g), optionally wherein the NGR motif at positions 54-56 of the heavy chain variable region is replaced with an SGR motif.

5. The antigen-binding fragment of any one of claims 1-4, comprising a Fab fragment.

6. The antigen-binding fragment of any one of claims 1-4, comprising a single chain antibody, wherein the heavy chain variable domain the light chain variable domain are joined together with a peptide linker.

7. The antigen-binding protein or fully human anti-CD38 antibody of any one of claims 1-4, which is an IgG1, IgG2, IgG3 or IgG4 class antibody.

8. The antigen-binding protein, antibody or antigen-binding fragment of any one of the preceding claims, that binds to CD38 proteins from human and cynomolgus.

9. The antigen-binding protein, antibody or antigen-binding fragment of any one of the preceding claims, that binds to cells expressing CD38 protein.

10. The antigen-binding protein, antibody or antigen-binding fragment of any one of the preceding claims, that binds to human myeloma cells expressing CD38 protein.

11. A pharmaceutical composition, comprising the antigen-binding protein, antibody or antigen-binding fragment of any one of the preceding claims and a pharmaceutically-acceptable excipient.

12. A first nucleic acid encoding a first polypeptide comprising the antibody heavy chain variable region of claim 3, wherein the amino acid sequence of the antibody heavy chain variable region has at least 95% sequence identity to the amino acid sequence of SEQ ID NO:3, 5, 6, 7, 9, 10, 11 or 13, and a second nucleic acid encoding a second polypeptide comprising the antibody light chain variable region of claim 2, wherein the amino acid sequence of the antibody light chain variable region has at least 95% sequence identity to the amino acid sequence of SEQ ID NO:4 or 12.

13. One or more nucleic acids encoding the antigen-binding protein, antibody or antigen-binding fragment of any one of claims 1-10.

14. One or more expression vectors comprising one or more promoters operably linked to the first and second nucleic acids of claim 12.

15. One or more expression vectors comprising one or more promoters operably linked to the one or more nucleic acids of claim 13.

16. A host cell harboring the one or more expression vectors of claim 14 or 15.

17. A method for preparing the first polypeptide comprising the antibody heavy chain variable region and the second polypeptide comprising the antibody light chain variable region or the antigen-binding protein, antibody or antigen-binding fragment, the method comprising: culturing a population of the host cell of claim 16 under conditions suitable for expressing the first polypeptide and the second polypeptide or the antibody or antigen-binding fragment.

18. The method of claim 17, further comprising: recovering from the population of the host cell the expressed first polypeptide and the expressed second polypeptide or the expressed antibody or antigen-binding fragment.

19. A method for killing CD38-expressing cells, comprising: contacting (i) a population of effector cells with (ii) a population of target cells which express CD38 (iii) in the presence of the antigen-binding protein, antibody or antigen-binding fragment of any one of claims 1-10, under conditions that are suitable for killing the CD38-expressing cells, optionally wherein the method is an in vitro method.

20. A method for treating a subject having a disease associated with CD38 over-expression or a CD38-positive cancer, the method comprising: administering to the subject an effective amount of a therapeutic composition comprising the antigen-binding protein, antibody or antigen-binding fragment of any one of claims 1-10.

21. A method for treating a subject having a CD38-positive cancer, wherein the CD38-positive cancer comprises a B-cell leukemia, B-cell lymphoma or B-cell myeloma, the method comprising: administering to the subject an effective amount of a therapeutic composition comprising the antigen-binding protein, antibody or antigen-binding fragment of any one of claims 1-10.

22. A method for treating a subject having a disease associated with CD38 expression, wherein the disease associated with CD38 expression is a multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CIVIL), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance, the method comprising: administering to the subject an effective amount of a therapeutic composition comprising the antigen-binding protein, antibody or antigen-binding fragment of any one of claims 1-10.


SEQUENCE LISTING (88 sequences)

SEQ ID NO: 1 - protein, 118 aa, Artificial Sequence
  Synthetic: A2 parental heavy chain, 3E10
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSS

SEQ ID NO: 2 - protein, 110 aa, Artificial Sequence
  Synthetic: A2 parental light chain, 3G3, 3E11, 3H10
QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL

SEQ ID NO: 3 - protein, 118 aa, Artificial Sequence
  Synthetic: 3H10m1, 3H10m1g
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS

SEQ ID NO: 4 - protein, 110 aa, Artificial Sequence
  Synthetic: 3H10m1, 3G8m1, 3D3m1, 3E10
QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 5 - protein, 118 aa, Artificial Sequence
  Synthetic: 3G8m1
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTNWGQGTLVTVSS

SEQ ID NO: 6 - protein, 118 aa, Artificial Sequence
  Synthetic: 3E3m1
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTDWGQGTLVTVSS

SEQ ID NO: 7 - protein, 118 aa, Artificial Sequence
  Synthetic: 3G3
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWSGEFTDWGQGTLVTVSS

SEQ ID NO: 8 - skipped

SEQ ID NO: 9 - protein, 118 aa, Artificial Sequence
  Synthetic: 3E11
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWGGEFTDWGQGTLVTVSS

SEQ ID NO: 10 - protein, 118 aa, Artificial Sequence; identical to SEQ ID NO: 3
  Synthetic: 3H10

SEQ ID NO: 11 - protein, 118 aa, Artificial Sequence
  Synthetic: 3H10N
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSS

SEQ ID NO: 12 - protein, 110 aa, Artificial Sequence
  Synthetic: 3H10N, 3H10NS , 3H10m1g
QSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 13 - protein, 118 aa, Artificial Sequence
  Synthetic: 3H10NS
QVQLVESGGGLVKPGGSLRLSCAASGFTFSDDYMSWIRQAPGKGLEWVASVSSGRPTTYYADSVRGRFTISRDNAKNSLYLQMNSLRAEDTAVYYCAREDWGGEFTDWGQGTLVTVSS

SEQ ID NO: 14 - protein, 330 aa, Artificial Sequence
  Synthetic: Anti-CD38 IgG1: heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 330 aa, Artificial Sequence
  Synthetic: Anti-CD38 IgG1-SPPC: heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 327 aa, Artificial Sequence
  Synthetic: Anti-CD38 IgG4: heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 106 aa, Artificial Sequence
  Synthetic: Anti-CD38: lambda light chain constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 107 aa, Artificial Sequence [sequence omitted]

SEQ ID NO: 19 - protein, 300 aa, Homo sapiens
  misc_feature: CD38 antigen - human
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 20 - protein, 300 aa, Macaca fascicularis
  misc_feature: CD38 antigen - cynomolgus monkey
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSG

SEQ ID NO: 21 - protein, 304 aa, Mus musculus
  misc_feature: CD38 antigen - mouse
MANYEFSQVSGDRPGCRLSRKAQIGLGVGLLVLIALVVGIVVILLRPRSLLVWTGEPTTKHFSDIFLGRCLIYTQILRPEMRDQNCQEILSTFKGAFVSKNPCNITREDYAPLVKLVTQTIPCNKTLFWSKSKHLAHQYTWIQGKMFTLEDTLLGYIADDLRWCGDPSTSDMNYVSCPHWSENCPNNPITVFWKVISQKFAEDACGVVQVMLNGSLREPFYKNSTFGSVEVFSLDPNKVHKLQAWVMHDIEGASSNACSSSSLNELKMIVQKRNMIFACVDNYRPARFLQCVKNPEHPSCRLNT

SEQ ID NO: 22 - protein, 303 aa, Rattus norvegicus
  misc_feature: CD38 antigen - rat
MANYEFSQVSEDRPGCRLTRKAQIGLGVGLLLLVALVVVVVIVLWPRSPLVWKGKPTTKHFADIILGRCLIYTQILRPEMRDQDCKKILSTFKRGFISKNPCNITNEDYAPLVKLVTQTIPCNKTLFWSKSKHLAHQYTWIQGKMFTLEDTLLGYIADDLRWCGDPSTSDMNYDSCPHWSENCPNNPVAVFWNVISQKFAEDACGVVQVMLNGSLSEPFYRNSTFGSVEVFNLDPNKVHKLQAWVMHDIKGTSSNACSSPSINELKSIVNKRNMIFACQDNYRPVRFLQCVKNPEHPSCRLNV

SEQ ID NO: 23 - protein, 5 aa, Artificial Sequence
  Synthetic: A2 (VH  CDR1)
DDYMS

SEQ ID NO: 24 - protein, 17 aa, Artificial Sequence
  Synthetic: A2 (VH  CDR2)
SVSNGRPTTYYADSVRG

SEQ ID NO: 25 - protein, 9 aa, Artificial Sequence
  Synthetic: A2 (VH  CDR3)
EDWGGEFTD

SEQ ID NO: 26 - protein, 13 aa, Artificial Sequence
  Synthetic: A2 (VL  CDR1)
SGSSSNIGINFVY

SEQ ID NO: 27 - protein, 7 aa, Artificial Sequence
  Synthetic: A2 (VL  CDR2)
KNNQRPS

SEQ ID NO: 28 - protein, 11 aa, Artificial Sequence
  Synthetic: A2 (VL  CDR3)
AAWDDSLSGYV

SEQ ID NO: 29 - protein, 5 aa, Artificial Sequence
  Synthetic: 3H10m1 (VH  CDR1)
DDYMS

SEQ ID NO: 30 - protein, 17 aa, Artificial Sequence
  Synthetic: 3H10m1 (VH  CDR2)
SVSNGRPTTYYADSVRG

SEQ ID NO: 31 - protein, 9 aa, Artificial Sequence
  Synthetic: 3H10m1 (VH  CDR3)
EGWSGEFTD

SEQ ID NO: 32 - protein, 13 aa, Artificial Sequence
  Synthetic: 3H10m1 (VL  CDR1)
SGSSSNIGFHFVY

SEQ ID NO: 33 - protein, 7 aa, Artificial Sequence
  Synthetic: 3H10m1 (VL  CDR2)
KNNQRPS

SEQ ID NO: 34 - protein, 11 aa, Artificial Sequence
  Synthetic: 3H10m1 (VL  CDR3)
AAWDDSLSGYV

SEQ ID NO: 35 - protein, 5 aa, Artificial Sequence
  Synthetic: 3G8m1 (VH  CDR1)
DDYMS

SEQ ID NO: 36 - protein, 17 aa, Artificial Sequence
  Synthetic: 3G8m1 (VH  CDR2)
SVSNGRPTTYYADSVRG

SEQ ID NO: 37 - protein, 9 aa, Artificial Sequence
  Synthetic: 3G8m1 (VH  CDR3)
EAWGGEFTN

SEQ ID NO: 38 - protein, 13 aa, Artificial Sequence
  Synthetic: 3G8m1 (VL  CDR1)
SGSSSNIGFHFVY

SEQ ID NO: 39 - protein, 7 aa, Artificial Sequence
  Synthetic: 3G8m1 (VL  CDR2)
KNNQRPS

SEQ ID NO: 40 - protein, 11 aa, Artificial Sequence
  Synthetic: 3G8m1 (VL  CDR3)
AAWDDSLSGYV

SEQ ID NO: 41 - protein, 5 aa, Artificial Sequence
  Synthetic: 3E3m1 (VH  CDR1)
DDYMS

SEQ ID NO: 42 - protein, 17 aa, Artificial Sequence
  Synthetic: 3E3m1 (VH  CDR2)
SVSNGRPTTYYADSVRG

SEQ ID NO: 43 - protein, 9 aa, Artificial Sequence
  Synthetic: 3E3m1 (VH  CDR3)
EAWGGEFTD

SEQ ID NO: 44 - protein, 13 aa, Artificial Sequence
  Synthetic: 3E3m1 (VL  CDR1)
SGSSSNIGFHFVY

SEQ ID NO: 45 - protein, 7 aa, Artificial Sequence
  Synthetic: 3E3m1 (VL  CDR2)
KNNQRPS

SEQ ID NO: 46 - protein, 11 aa, Artificial Sequence
  Synthetic: 3E3m1 (VL  CDR3)
AAWDDSLSGYV

SEQ ID NO: 47 - protein, 5 aa, Artificial Sequence
  Synthetic: 3G3 (VH  CDR1)
DDYMS

SEQ ID NO: 48 - protein, 17 aa, Artificial Sequence
  Synthetic: 3G3 (VH  CDR2)
SVSNGRPTTYYADSVRG

SEQ ID NO: 49 - protein, 9 aa, Artificial Sequence
  Synthetic: 3G3 (VH  CDR3)
EAWSGEFTD

SEQ ID NO: 50 - protein, 13 aa, Artificial Sequence
  Synthetic: 3G3 (VL  CDR1)
SGSSSNIGINFVY

SEQ ID NO: 51 - protein, 7 aa, Artificial Sequence
  Synthetic: 3G3 (VL  CDR2)
KNNQRPS

SEQ ID NO: 52 - protein, 11 aa, Artificial Sequence
  Synthetic: 3G3 (VL  CDR3)
AAWDDSLSGYV

SEQ ID NO: 53 - protein, 5 aa, Artificial Sequence
  Synthetic: 3E11(VH  CDR1)
DDYMS

SEQ ID NO: 54 - protein, 17 aa, Artificial Sequence
  Synthetic: 3E11(VH  CDR2)
SVSNGRPTTYYADSVRG

SEQ ID NO: 55 - protein, 9 aa, Artificial Sequence
  Synthetic: 3E11(VH  CDR3)
EGWGGEFTD

SEQ ID NO: 56 - protein, 13 aa, Artificial Sequence
  Synthetic: 3E11(VL  CDR1)
SGSSSNIGINFVY

SEQ ID NO: 57 - protein, 7 aa, Artificial Sequence
  Synthetic: 3E11(VL  CDR2)
KNNQRPS

SEQ ID NO: 58 - protein, 11 aa, Artificial Sequence
  Synthetic: 3E11(VL  CDR3)
AAWDDSLSGYV

SEQ ID NO: 59 - protein, 5 aa, Artificial Sequence
  Synthetic: 3H10 (VH  CDR1)
DDYMS

SEQ ID NO: 60 - protein, 17 aa, Artificial Sequence
  Synthetic: 3H10 (VH  CDR2)
SVSNGRPTTYYADSVRG

SEQ ID NO: 61 - protein, 9 aa, Artificial Sequence
  Synthetic: 3H10 (VH  CDR3)
EGWSGEFTD

SEQ ID NO: 62 - protein, 13 aa, Artificial Sequence
  Synthetic: 3H10 (VL  CDR1)
SGSSSNIGINFVY

SEQ ID NO: 63 - protein, 7 aa, Artificial Sequence
  Synthetic: 3H10 (VL  CDR2)
KNNQRPS

SEQ ID NO: 64 - protein, 11 aa, Artificial Sequence
  Synthetic: 3H10 (VL  CDR3)
AAWDDSLSGYV

SEQ ID NO: 65 - protein, 5 aa, Artificial Sequence
  Synthetic: 3H10N (VH  CDR1)
DDYMS

SEQ ID NO: 66 - protein, 17 aa, Artificial Sequence
  Synthetic: 3H10N (VH  CDR2)
SVSNGRPTTYYADSVRG

SEQ ID NO: 67 - protein, 9 aa, Artificial Sequence
  Synthetic: 3H10N (VH  CDR3)
EDWGGEFTD

SEQ ID NO: 68 - protein, 13 aa, Artificial Sequence
  Synthetic: 3H10N (VL  CDR1)
SGSSSNIGFHFVY

SEQ ID NO: 69 - protein, 7 aa, Artificial Sequence
  Synthetic: 3H10N (VL  CDR2)
KNNQRPS

SEQ ID NO: 70 - protein, 11 aa, Artificial Sequence
  Synthetic: 3H10N (VL  CDR3)
AAWDDSLSGYV

SEQ ID NO: 71 - protein, 5 aa, Artificial Sequence
  Synthetic: 3H10NS (VH  CDR1)
DDYMS

SEQ ID NO: 72 - protein, 17 aa, Artificial Sequence
  Synthetic: 3H10NS (VH  CDR2)
SVSSGRPTTYYADSVRG

SEQ ID NO: 73 - protein, 9 aa, Artificial Sequence
  Synthetic: 3H10NS (VH  CDR3)
EDWGGEFTD

SEQ ID NO: 74 - protein, 13 aa, Artificial Sequence
  Synthetic: 3H10NS (VL  CDR1)
SGSSSNIGFHFVY

SEQ ID NO: 75 - protein, 7 aa, Artificial Sequence
  Synthetic: 3H10NS (VL  CDR2)
KNNQRPS

SEQ ID NO: 76 - protein, 11 aa, Artificial Sequence
  Synthetic: 3H10NS (VL  CDR3)
AAWDDSLSGYV

SEQ ID NO: 77 - protein, 5 aa, Artificial Sequence
  Synthetic: 3E10 (VH  CDR1)
DDYMS

SEQ ID NO: 78 - protein, 17 aa, Artificial Sequence
  Synthetic: 3E10 (VH  CDR2)
SVSNGRPTTYYADSVRG

SEQ ID NO: 79 - protein, 9 aa, Artificial Sequence
  Synthetic: 3E10 (VH  CDR3)
EDWGGEFTD

SEQ ID NO: 80 - protein, 13 aa, Artificial Sequence
  Synthetic: 3E10 (VL  CDR1)
SGSSSNIGFHFVY

SEQ ID NO: 81 - protein, 7 aa, Artificial Sequence
  Synthetic: 3E10 (VL  CDR2)
KNNQRPS

SEQ ID NO: 82 - protein, 11 aa, Artificial Sequence
  Synthetic: 3E10 (VL  CDR3)
AAWDDSLSGYV

SEQ ID NO: 83 - protein, 5 aa, Artificial Sequence
  Synthetic: 3H10m1g (VH  CDR1)
DDYMS

SEQ ID NO: 84 - protein, 17 aa, Artificial Sequence
  Synthetic: 3H10m1g (VH  CDR2)
SVSNGRPTTYYADSVRG

SEQ ID NO: 85 - protein, 9 aa, Artificial Sequence
  Synthetic: 3H10m1g (VH  CDR3)
EGWSGEFTD

SEQ ID NO: 86 - protein, 13 aa, Artificial Sequence
  Synthetic: 3H10m1g (VL  CDR1)
SGSSSNIGFHFVY

SEQ ID NO: 87 - protein, 7 aa, Artificial Sequence
  Synthetic: 3H10m1g (VL  CDR2)
KNNQRPS

SEQ ID NO: 88 - protein, 11 aa, Artificial Sequence
  Synthetic: 3H10m1g (VL  CDR3)
AAWDDSLSGYV